(19) United States
(12) Patent Application Publication
(10) Pub. No.: US 2024/0399019 A1
(43) Pub. Date: Dec. 5, 2024

(54) ADHESIVE NONFIBROTIC INTERFACES AND METHODS

(71) Applicant: Massachusetts Institute of Technology, Cambridge, MA (US)

(72) Inventors: Jingjing Wu, Cambridge, MA (US); Jue Deng, Cambridge, MA (US); Hyunwoo Yuk, Cambridge, MA (US); Xuanhe Zhao, Allston, MA (US)

(21) Appl. No.: 18/606,479

(22) Filed: Mar. 15, 2024

Related U.S. Application Data
(60) Provisional application No. 63/490,669, filed on Mar. 16, 2023.

(51) Int. Cl. A61L 24/00; A61L 24/10; A61N 1/05

(52) U.S. Cl. CPC A61L 24/0031; A61L 24/102; A61N 1/05

(57) ABSTRACT

Methods of implanting biomaterials and devices in a patient, such as methods that include arranging an adhesive composition between and in contact with a biomaterial or a device, and a surface of a tissue of the patient. An adhesive composition may be configured to form at a surface of a tissue a conformal interface that reduces or minimizes infiltration of inflammatory cells at the conformal interface.

### CROSS-REFERENCE TO RELATED APPLICATIONS

This application claims priority to U.S. Provisional Patent Application No. 63/490,669, filed Mar. 16, 2023, which is incorporated by reference herein.

### STATEMENT REGARDING FEDERALLY SPONSORED RESEARCH OR DEVELOPMENT

This invention was made with government support under contract number 1-R01-HL153857-01 awarded by National Institutes of Health, and contract number EFMA-1935291 awarded by the National Science Foundation. The government has certain rights in this invention.

### BACKGROUND

Foreign body reaction to implants can undermine the long-term functionality and reliability of biomaterials and devices in vivo. In particular, the formation of a fibrous capsule between an implant and a target tissue, as a result of foreign body reaction, can compromise an implant's efficacy, usually because the fibrous capsule acts as a barrier for mechanical, electrical, chemical, optical communications, or a combination thereof. (See, e.g., Chandorkar, Y. et al., ACS Biomaterials Science & Engineering 5, 19-44 (2018)).

To alleviate the fibrous capsule formation at the implant-tissue interface, various approaches have been developed, such as drug-eluting coatings (see, e.g., Farah, S. et al., Nature Materials 18, 892-904 (2019), hydrophilic polymer coatings (see, e.g., Gudipati, C. S. et al., Langmuir 21, 3044-3053 (2005)), zwitterionic polymer coatings (see, e.g., Zhang, L. et al., Nature Biotechnology 31, 553-556 (2013)), active surfaces (see, e.g., Dolan, E. B. et al., Science Robotics 4, eaax7043 (2019)), controlled stiffness (see, e.g., Noskovicova, N. et al. Nature Biomedical Engineering 5, 1437-1456 (2021)), and the size of the implant (see, e.g., Veiseh, O. et al., Nature Materials 14, 643-651 (2015)).

Despite these efforts, the mitigation of fibrous capsule formation for implanted biomaterials and devices remains an ongoing challenge in the field.

There remains a need for new solutions and strategies for improving foreign body reaction, such as by preventing or lessening the formation of a fibrous capsule.

### BRIEF SUMMARY

Provided herein are methods of implantation that may delay or reduce the likelihood of the formation of a fibrous capsule.

In one aspect, methods of implantation are provided, such as implanting a biomaterial or a device in a patient. In some embodiments, the methods include arranging an adhesive composition between and in contact with a biomaterial or a device, and a surface of a tissue of the patient. The adhesive composition may be configured to form at the surface of the tissue a conformal interface that reduces or minimizes infiltration of inflammatory cells at the conformal interface. In some embodiments, the reduction or the minimization of the infiltration of inflammatory cells delays the formation of a fibrous capsule at the conformal interface. For example, the reduction or minimization of an infiltration of inflammatory cells may prevent the formation of a fibrous capsule at a conformal interface for at least 28 days, at least 60 days, or at least 84 days after the implanting of the biomaterial or the device.

In another aspect, compositions are provided, such as adhesive compositions. In some embodiments, the adhesive compositions are configured to form at a surface of a tissue a conformal interface that reduces or minimizes infiltration of inflammatory cells at the conformal interface.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1A is a schematic illustration of an embodiment of a non-adhesive implant that includes a mock device and a non-adhesive layer.

FIG. 1B is a schematic illustration of an embodiment of an in vivo implantation with fibrous capsule formation at an implant tissue interface.

FIG. 1C is a schematic illustration of an embodiment of an adhesive implant that includes a mock device and an embodiment of an adhesive layer.

FIG. 1D is a schematic illustration of an embodiment of an in vivo implantation without fibrous capsule formation at the implant tissue interface.

FIG. 1E is a histology image of native tissue (left), an embodiment of an adhesive implant (middle), and an embodiment of a non-adhesive implant (right) collected 28 days after implantation to the abdominal wall.

FIG. 1F is a histology image of native tissue (left), an embodiment of an adhesive implant (middle), and an embodiment of a non-adhesive implant (right) collected 28 days after implantation to the colon.

FIG. 1G is a histology image of native tissue (left), an embodiment of an adhesive implant (middle), and an embodiment of a non-adhesive implant (right) collected 28 days after implantation to the stomach.

FIG. 1H is a histology image of native tissue (left), an embodiment of an adhesive implant (middle), and an embodiment of a non-adhesive implant (right) collected 28 days after implantation to the lung.

FIG. 1I is a histology image of native tissue (left), an embodiment of an adhesive implant (middle), and an embodiment of a non-adhesive implant (right) collected 28 days after implantation to the heart.

FIG. 2A is a schematic of various animal studies described herein.

FIG. 2B is a series of photographs of various organs collected 28 days after implantation of an embodiment of a non-adhesive implant.

FIG. 2C is a series of photographs of various organs collected 28 days after implantation of an embodiment of an adhesive implant.

FIG. 3A is a series of histology images stained with Masson's trichrome (left) and hematoxylin and eosin stain (right) of an embodiment of an adhesive implant collected 28 days after implantation to an abdominal wall.

FIG. 3B is a series of histology images stained with Masson's trichrome (left) and hematoxylin and eosin stain (right) of an implant-tissue interface for an embodiment of an adhesive implant collected 28 days after implantation to an abdominal wall.

FIG. 3C is a series of histology images stained with Masson's trichrome (left) and hematoxylin and eosin stain (right) of an implant-cavity interface for an embodiment of an adhesive implant collected 28 days after implantation to an abdominal wall.

FIG. 4A is a histology image stained with Masson's trichrome (left) and hematoxylin and eosin stain (right) of a native abdominal wall tissue without an implant collected 84 days after surgery.

FIG. 4B is a histology image stained with Masson's trichrome (left) and hematoxylin and eosin stain (right) of an embodiment of an adhesive implant collected 84 days after implantation to an abdominal wall.

FIG. 5 is a tunneling electron microscopy (TEM) image of an embodiment of an adhesive implant-tissue interface.

FIG. 6A is a histology image stained with Masson's trichrome of an embodiment of a polyurethane mock device collected 28 days after implantation to an abdominal wall.

FIG. 6B is a histology image stained with hematoxylin and eosin stain of an embodiment of a polyurethane mock device collected 28 days after implantation to an abdominal wall.

FIG. 7A is a histology image stained with Masson's trichrome (left) and hematoxylin and eosin (right) of an embodiment of a non-adhesive implant collected 3 days after implantation to an abdominal wall.

FIG. 7B is a histology image stained with Masson's trichrome (left) and hematoxylin and eosin (right) of an embodiment of a non-adhesive implant collected 7 days after implantation to an abdominal wall.

FIG. 7C is a histology image stained with Masson's trichrome (left) and hematoxylin and eosin (right) of an embodiment of a non-adhesive implant collected 14 days after implantation to an abdominal wall.

FIG. 7D is a histology image stained with Masson's trichrome (left) and hematoxylin and eosin (right) of an embodiment of a non-adhesive implant collected 28 days after implantation to an abdominal wall.

FIG. 7E is a histology image stained with Masson's trichrome (left) and hematoxylin and eosin (right) of an embodiment of an adhesive implant collected 3 days after implantation to an abdominal wall.

FIG. 7F is a histology image stained with Masson's trichrome (left) and hematoxylin and eosin (right) of an embodiment of an adhesive implant collected 7 days after implantation to an abdominal wall.

FIG. 7G is a histology image stained with Masson's trichrome (left) and hematoxylin and eosin (right) of an embodiment of an adhesive implant collected 14 days after implantation to an abdominal wall.

FIG. 7H is a histology image stained with Masson's trichrome (left) and hematoxylin and eosin (right) of an embodiment of an adhesive implant collected 28 days after implantation to an abdominal wall.

FIG. 7I depicts collagen layer thicknesses observed for native tissue, an embodiment of a non-adhesive interface, and an embodiment of an adhesive interface.

FIG. 8A is a schematic illustration of an experimental setup for an in vitro protein adsorption assay for an embodiment of a non-adhesive implant.

FIG. 8B is a confocal microscope image of an in vitro albumin adsorption assay for an embodiment of a non-adhesive implant.

FIG. 8C is a confocal microscope image of an in vitro fibrinogen adsorption assay for the non-adhesive implant.

FIG. 8D is a schematic illustration of an experimental setup for an in vitro protein adsorption assay for an embodiment of an adhesive implant.

FIG. 8E is a confocal microscope image of an in vitro albumin adsorption assay for an embodiment of an adhesive implant.

FIG. 8F is a confocal microscope image of an in vitro fibrinogen adsorption assay for an embodiment of an adhesive implant.

FIG. 8G depicts relative fluorescence intensities at an embodiment of an implant-substrate interface measured 30 minutes after co-culture for albumin.

FIG. 8H depicts relative fluorescence intensities at an embodiment of an implant-substrate interface measured 30 minutes after co-culture for fibrinogen.

FIG. 9A is an immunofluorescence image of an embodiment of a non-adhesive implant collected 3 days after implantation to an abdominal wall.

FIG. 9B is an immunofluorescence image of an embodiment of an adhesive implant collected 3 days after implantation to an abdominal wall.

FIG. 9C is an immunofluorescence image of an embodiment of a non-adhesive implant collected 7 days after implantation to an abdominal wall.

FIG. 9D is an immunofluorescence image of an embodiment of an adhesive implant collected 7 days after implantation to an abdominal wall.

FIG. 9E is an immunofluorescence image of an embodiment of a non-adhesive implant collected 14 days after implantation to an abdominal wall.

FIG. 9F is an immunofluorescence image of an embodiment of an adhesive implant collected 14 days after implantation to an abdominal wall.

FIG. 9G depicts normalized fluorescence intensity from immunofluorescence images at 3 days after implantation of an embodiment of a device.

FIG. 9H depicts normalized fluorescence intensity from immunofluorescence images at 7 days after implantation of an embodiment of a device.

FIG. 9I depicts normalized fluorescence intensity from immunofluorescence images at 14 days after implantation of an embodiment of a device.

FIG. 10A is an immunofluorescence image of an embodiment of a non-adhesive implant collected 28 days after implantation to an abdominal wall.

FIG. 10B is an immunofluorescence image of an embodiment of an adhesive implant collected 28 days after implantation to an abdominal wall.

FIG. 10C depicts normalized fluorescence intensity from immunofluorescence images 28 days after implantation of an embodiment of a device.

FIG. 11A is a schematic showing cytokines and genes relevant to each cell type in embodiments of studies described herein.

FIG. 11B is a heatmap of immune response-related cytokines and chemokines measured with an assay of an embodiment of a non-adhesive implant-tissue interface and an embodiment of an adhesive implant-tissue interface collected 1, 3, 7, and 14 days after implantation to an abdominal wall.

FIG. 11C depicts normalized gene expression of immune response-related markers for embodiments of a non-adhesive implant-tissue interface and an embodiment of an adhesive implant-tissue interface collected 1 day after implantation to an abdominal wall.

FIG. 11D depicts normalized gene expression of immune response-related markers for embodiments of a non-adhesive implant-tissue interface and an embodiment of an adhesive implant-tissue interface collected 3 days after implantation to an abdominal wall.

FIG. 11E depicts normalized gene expression of immune response-related markers for embodiments of a non-adhesive implant-tissue interface and an embodiment of an adhesive implant-tissue interface collected 7 days after implantation to an abdominal wall.

FIG. 11F depicts normalized gene expression of immune response-related markers for embodiments of a non-adhesive implant-tissue interface and an embodiment of an adhesive implant-tissue interface collected 14 days after implantation to an abdominal wall.

FIG. 12A depicts immunofluorescence images of an embodiment of an adhesive implant collected 3 days after implantation to an abdominal wall.

FIG. 12B depicts a quantification of iNOS+/Neutrophil Elastase+ and iNOS+/CD68+ cells per unit area 3 days after implantation of an embodiment of a device.

FIG. 13A is a principal component analysis (PCA) plot depicting the variances of an embodiment of an adhesive implant-tissue interface dataset and an embodiment of a non-adhesive implant-tissue interface dataset collected 3 days after implantation to the abdominal wall.

FIG. 13B is a volcano plot displaying gene expression profiles when comparing embodiments of adhesive and non-adhesive implant-tissue interfaces collected 3 days after implantation to the abdominal wall.

FIG. 13C depicts a top 5 enriched processes from Gene Ontology (GO) enrichment analysis of differentially expressed genes in embodiments of non-adhesive and adhesive implant-tissue interfaces collected 3 days after implantation to an abdominal wall.

FIG. 13D is a PCA plot illustrating variances of embodiments of adhesive and non-adhesive implant-tissue interface datasets collected 14 days after implantation to an abdominal wall.

FIG. 13E is a volcano plot depicts gene expression profiles when comparing embodiments of adhesive and non-adhesive implant-tissue interfaces collected 14 days after implantation.

FIG. 13F depicts top 5 enriched processes from GO enrichment analysis of differently expressed genes in embodiments of non-adhesive and adhesive implant-tissue interfaces collected 14 days after implantation to an abdominal wall.

FIG. 14A depicts a bi-clustering heatmap visualizing the expression profiles of the top 30 differentially expressed genes sorted by their adjusted P value by plotting their log2 transformed expression values in samples 3 days after implantation of an embodiment of a device.

FIG. 14B depicts a bi-clustering heatmap visualizing the expression profiles of the top 30 differentially expressed genes sorted by their adjusted P value by plotting their log2 transformed expression values in samples 14 days after implantation of an embodiment of a device.

FIG. 15A is a schematic illustration of an embodiment of a study design in C57BL/6 mice.

FIG. 15B includes representative histology images stained with Masson's trichrome (MTS) and hematoxylin and eosin (H&E) for native tissue (left), an embodiment of an adhesive implant (middle), and an embodiment of a non-adhesive implant (right) collected on day 28 post-implantation in C57BL/6 mice.

FIG. 15C is a schematic illustration of an embodiment of a study design in HuCD34-NCG humanized mice.

FIG. 15D includes representative histology images stained with MTS and H&E for native tissue (left), an embodiment of an adhesive implant (middle), and an embodiment of a non-adhesive implant (right) collected on day 28 post-implantation in HuCD34-NCG humanized mice.

FIG. 15E is a schematic illustration of an embodiment of a study design in HuCD34-NCG humanized pigs.

FIG. 15F includes representative histology images stained with MTS and H&E for native tissue (left), an embodiment of an adhesive implant (middle), and an embodiment of a non-adhesive implant (right) collected on day 7 post-implantation in pigs.

FIG. 16A is a schematic illustration for in vivo electrophysiological recording and stimulation via an embodiment of implanted electrodes with embodiments of non-adhesive and adhesive implant-tissue interfaces.

FIG. 16B depicts recorded R-wave amplitudes via implanted electrodes with embodiments of adhesive and non-adhesive implant-tissue interfaces on days 0, 3, 7, 14, and 28 after implantation to a rat heart.

FIG. 16C depicts an epicardial electrocardiogram after stimulation via implanted electrodes with an embodiment of a non-adhesive implant-tissue interface on days 0, 3, 7, 14, and 28 after implantation to a rat heart.

FIG. 16D depicts an epicardial electrocardiogram after stimulation via implanted electrodes with an embodiment of an adhesive implant-tissue interface on days 0, 3, 7, 14, and 28 after implantation to a rat heart.

FIG. 16E depicts histology images stained with hematoxylin and eosin and Masson's trichrome of an embodiment of a non-adhesive implant collected 28 days after implantation to a rat heart.

FIG. 16F depicts histology images stained with hematoxylin and eosin and Masson's trichrome of an embodiment of an adhesive implant collected 28 days after implantation to a rat heart.

### DETAILED DESCRIPTION

In some embodiments, the methods herein include methods of implanting a biomaterial or a device, e.g., a medical device, into the body of patient. The patient may be a human or other mammal. The methods may include arranging an adhesive composition, such as those described herein, between and in contact with (i) a biomaterial or a device, and (ii) a surface of a tissue of a patient.

The arranging of an adhesive composition may be achieved by any known technique. For example, the methods may include providing an adhesive composition that is affixed to a biomaterial or a device; and then contacting a tissue, such as a surface of a tissue, with the adhesive composition. As a further example, the methods may include providing a biomaterial or a device, affixing an adhesive composition to the biomaterial or the device, and then contacting a tissue, such as a surface of a tissue, with the adhesive composition. As yet another example, the methods may include disposing an adhesive composition on a tissue, such as a surface of the tissue, and contacting the adhesive composition with the biomaterial or the device.

In particular embodiments, the adhesive composition advantageously is configured to form at the surface of the tissue a conformal interface that reduces or minimizes infiltration of inflammatory cells at the conformal interface. The reduction or the minimization of the infiltration of inflammatory cells may prevent the formation of a fibrous capsule at the conformal interface for at least 28 days, at least 30 days, at least 40 days, at least 50 days, at least 60 days, at least 70 days, at least 80 days, at least 84 days, at least 90 days, or at least 100 days after the implanting of the biomaterial or the device.

### Adhesive Compositions

Any adhesive composition capable of forming a conformal interface may be used in the methods provided herein. The adhesive compositions may be a polymeric composition. The phrase “polymeric composition” refers to and includes any composition that includes or consists of a polymeric compound. A “polymeric compound” is a compound formed by the bonding together of two or more monomers (e.g., three or more monomers, four or more monomers, etc.); therefore, a “polymeric compound” may be a polymer, a copolymer (i.e., a polymer formed of two or more different types of monomer), oligomer, etc., or a combination thereof. Non-limiting examples of adhesive compositions that may be used in some embodiments of the methods described herein are disclosed by U.S. Patent Application Publication No. 2020/0353120 A1, which is incorporated by reference herein.

The polymeric composition may be a crosslinked polymeric composition. A polymeric composition is “crosslinked” when it includes a crosslinker bonded to two or more monomers, wherein the two or more monomers bonded to the crosslinker are intermolecular monomers, intramolecular monomers, or a combination thereof.

The polymer composition may include one or more hydrophilic polymers, one or more zwitterionic polymers, or a combination thereof. The one or more hydrophilic polymers may include any of those known in the art. Non-limiting examples of hydrophilic polymers include polyethylene glycol, polyacrylic acid, polyacrylamide, polyvinyl alcohol, polyhydroxy ethyl methacrylate, polyethylene glycol, polyurethane, casein, albumin, gelatin, chitosan, dextran, hyaluronic acid, alginate, cellulose, polyvinyl pyrrolidone, polystyrene sulfonate, collagen, alginic acid, pectin, oxidized derivatives thereof, and combinations thereof. For example, the alginate and the cellulose may be oxidized alginate and oxidized cellulose, respectively.

The one or more zwitterionic polymers may include any of those known in the art. Non-limiting examples of zwitterionic polymers include poly(phospobetaine), poly(carboxybetaine), poly(sulfobetaine), and a combination thereof.

The adhesive compositions may include one or more tissue coupling groups. As used herein, the phrase “tissue coupling groups” refers to a functional group or a compound that includes a functional group that is capable of reacting with one or more tissues, e.g., via one or more functional groups of a tissue, such as a functional group at or near a surface of a tissue, e.g., an amine. Non-limiting examples of tissue coupling groups include an amine coupling group, a thiol coupling group, a cysteine coupling group, an N-acetyl-cysteine coupling group, a boronate ester coupling group, and a combination thereof.

Non-limiting examples of amine coupling groups include N-hydroxysuccinimide ester (PAAc-co-NHS ester), a PAAm-co-NHS, an N-Hydroxysuccinimide (NHS) PEG, a poly(L-lactide-co-glycolide)-NHS, a poly(D,L-lactide)-polyethylene glycol-CO-NHS, a poly(N-isopropylacrylamide) N-hydroxysuccinimide terminated, an aldehyde, an imidoester, an epoxide, an isocyanate, a catechol, and a combination thereof. Non-limiting examples of thiol coupling groups include alginate, albumin, fibrinogen, collagen, chitosan, gelatin, and a combination thereof. Non-limiting examples of cysteine coupling groups include fibrinogen, collagen, and a combination thereof. Non-limiting examples of boronate ester coupling groups include acrylamide, N-isopropylacrylamide, polyvinyl alcohol (PVA), alginate, cellulose, and a combination thereof.

The adhesive compositions may include one or more crosslinkers. Non-limiting examples of crosslinkers include gelatin methacrylate, hyaluronic acid methacrylate, oxidized methacrylic alginate, polycaprolactone diacrylate, N,N′-bis(acryloyl) cystamine, N,N′-methylenebis(acrylamide), polyethylene glycol diacrylate, polyethylene glycol dimethacrylate, and a combination thereof.

### Tissues

A tissue of the methods described herein generally may include any tissue of a patient. The tissue may be an external tissue, an internal tissue, or a combination thereof.

The tissue may be an organ tissue, such as a surface of an organ. In some embodiments, the tissue includes abdominal wall tissue, colon tissue, stomach tissue, lung tissue, or heart tissue.

### Devices and Biomaterials

The biomaterials generally may include any material or substance that imparts a medical benefit to a patient, such as treatment, prophylaxis, etc. The biomaterials may be natural, synthetic, or a combination thereof. The biomaterials may include regenerative biomaterials. The biomaterials may include polymers, ceramics, inorganic glasses, or a combination thereof. The devices generally may include any known devices, such as any implantable device that imparts a medical benefit to a patient, such as treatment, prophylaxis, etc. In some embodiments, the device includes an electrode.

In some embodiments, the methods described herein allow bi-directional electrical communication between a device, such as an electrode, and a tissue to be maintained for a desirable time after implantation of the device in a patient. For example, bi-directional electrical communication between a device, such as an electrode, and a tissue may be maintained for at least 10 days, at least 15 days, at least 20 days, at least 25 days, at least 28 days, at least 30 days, at least 35 days, at least 40 days, at least 45 days, at least 50 days after the implanting of the device.

For example, the tissue may include heart tissue of the patient's heart, and an R-wave of the patient's heart recorded by the electrode at least 10 days, at least 15 days, at least 20 days, at least 25 days, at least 28 days, at least 30 days, at least 35 days, at least 40 days, at least 45 days, at least 50 days after the implanting of an electrode may have an amplitude that is 0% to about 5% less than an initial amplitude of an initial R-wave of the patient's heart recorded by the electrode within 24 hours after the implanting of the electrode. As a further example, the tissue may include heart tissue of the patient's heart, and a minimal stimulation current pulse amplitude for pacing emitted by the electrode may pace the patient's heart for at least 10 days, at least 15 days, at least 20 days, at least 25 days, at least 28 days, at least 30 days, at least 35 days, at least 40 days, at least 45 days, at least 50 days after the implanting of the electrode.

All referenced publications are incorporated by reference. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of various embodiments, applicants in no way disclaim these technical aspects, and it is contemplated that the present disclosure may encompass one or more of the conventional technical aspects discussed herein.

The present disclosure may address one or more of the problems and deficiencies of known methods and processes. However, it is contemplated that various embodiments may prove useful in addressing other problems and deficiencies in a number of technical areas. Therefore, the present disclosure should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

In the descriptions provided herein, the terms “includes,” “is,” “containing,” “having,” and “comprises” are used in an open-ended fashion, and thus should be interpreted to mean “including, but not limited to.” When methods, devices, compositions, materials, etc. are claimed or described in terms of “comprising” various steps or components, the methods, devices, compositions, materials, etc. can also “consist essentially of” or “consist of” the various steps or components, unless stated otherwise.

The terms “a,” “an,” and “the” are intended to include plural alternatives, e.g., at least one. For instance, the disclosure of “a hydrophilic polymer”, “a device”, and the like, is meant to encompass one, or mixtures or combinations of more than one hydrophilic polymer, device, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate.

As used herein, the term “about” means plus or minus 10% of the numerical value of the number with which it is being used.

### EMBODIMENTS

The following is a non-limiting listing of embodiments of the disclosure.

Embodiment 1. A method of implanting a biomaterial or a device in a patient, the method comprising, consisting essentially of, or consisting of arranging an adhesive composition between and in contact with (i) the biomaterial or the device, and (ii) a surface of a tissue of the patient.

Embodiment 2. The method of Embodiment 1, wherein the patient is a human or mammal.

Embodiment 3. The method of Embodiment 1 or 2, wherein the adhesive composition is configured to form at the surface of the tissue a conformal interface that reduces or minimizes infiltration of inflammatory cells at the conformal interface.

Embodiment 4. The method of Embodiment 3, wherein the reduction or the minimization of the infiltration of inflammatory cells prevents the formation of a fibrous capsule at the conformal interface for at least 28 days, at least 30 days, at least 40 days, at least 50 days, at least 60 days, at least 70 days, at least 80 days, at least 84 days, at least 90 days, or at least 100 days after the implanting of the biomaterial or the device.

Embodiment 5. The method of any of the preceding Embodiments, wherein the arranging of the adhesive composition comprises, consisting essentially of, or consisting of providing the adhesive composition, wherein the adhesive composition is affixed to the biomaterial or the device; and contacting the surface of the tissue with the adhesive composition.

Embodiment 6. The method of any of Embodiments 1 to 5, wherein the arranging of the adhesive composition comprises, consists essentially of, or consists of disposing the adhesive composition on the surface of the tissue; and contacting the adhesive composition with the biomaterial or the device.

Embodiment 7. An adhesive composition configured to form at a surface of a tissue a conformal interface that reduces or minimizes infiltration of inflammatory cells at the conformal interface.

### Biomaterials

Embodiment 8. The method or adhesive composition of any of the preceding Embodiments, wherein the biomaterial comprises, consists essentially of, or consists of a material or substance that imparts a medical benefit to a patient, such as treatment, prophylaxis, etc.

Embodiment 9. The method or adhesive composition of any of the preceding Embodiments, wherein the biomaterial is natural, synthetic, or a combination thereof.

Embodiment 10. The method or adhesive composition of any of the preceding Embodiments, wherein the biomaterial comprises, consists essentially of, or consists of a regenerative biomaterial.

Embodiment 11. The method or adhesive composition of any of the preceding Embodiments, wherein the biomaterial comprises, consists essentially of, or consists of a polymer, a ceramic, an inorganic glass, or a combination thereof.

### Devices

Embodiment 12. The method or adhesive composition of any of the preceding Embodiments, wherein the device comprises, consists essentially of, or consists of an implantable device.

Embodiment 13. The method or adhesive composition of any of the preceding Embodiments, wherein the device imparts a medical benefit to the patient, such as treatment, prophylaxis, etc.

Embodiment 14. The method or adhesive composition of any of the preceding Embodiments, wherein the device comprises, consists essentially of, or consists of an electrode.

### Electrical Communication

Embodiment 15. The method or adhesive composition of any of the preceding Embodiments, wherein communication, such as electrical communication (e.g., bi-directional electrical communication) between the device (or part thereof, e.g., the electrode) and the tissue is maintained for at least 10 days, at least 15 days, at least 20 days, at least 25 days, at least 28 days, at least 30 days, at least 35 days, at least 40 days, at least 45 days, at least 50 days after the implanting of the device.

### Tissues

Embodiment 16. The method or adhesive composition of any of the preceding Embodiments, wherein the tissue comprises, consists essentially of, or consists of an external tissue, an internal tissue, or a combination thereof.

Embodiment 17. The method or adhesive composition of any of the preceding Embodiments, wherein the tissue comprises, consists essentially of, or consists of abdominal wall tissue, colon tissue, stomach tissue, lung tissue, or heart tissue.

Embodiment 18. The method or adhesive composition of any of the preceding Embodiments, wherein the tissue comprises, consists essentially of, or consists of heart tissue of the patient's heart, and an R-wave of the patient's heart recorded by the electrode at least 28 days after the implanting of the electrode has an amplitude that is 0% to about 5% less than an initial amplitude of an initial R-wave of the patient's heart recorded by the electrode within 24 hours after the implanting of the electrode.

Embodiment 19. The method or adhesive composition of any of the preceding Embodiments, wherein the tissue comprises, consists essentially of, or consists of heart tissue of the patient's heart, and an R-wave of the patient's heart recorded by the electrode at least 10 days, at least 15 days, at least 20 days, at least 25 days, at least 28 days, at least 30 days, at least 35 days, at least 40 days, at least 45 days, at least 50 days after the implanting of an electrode has an amplitude that is 0% to about 5% less than an initial amplitude of an initial R-wave of the patient's heart recorded by the electrode within 24 hours after the implanting of the electrode.

Embodiment 20. The method or adhesive composition of any of the preceding Embodiments, wherein the tissue comprises, consists essentially of, or consists of heart tissue of the patient's heart, and a minimal stimulation current pulse amplitude for pacing emitted by the electrode may pace the patient's heart for at least 10 days, at least 15 days, at least 20 days, at least 25 days, at least 28 days, at least 30 days, at least 35 days, at least 40 days, at least 45 days, at least 50 days after the implanting of the electrode.

### Adhesive Compositions

Embodiment 21. The method or adhesive composition of any of the preceding Embodiments, wherein the adhesive composition comprises, consists essentially of, or consists of any one or more of the adhesive compositions disclosed by WO 2020231559A1, or U.S. Patent Application Publication No. 2020/0353120 A1, which are incorporated by reference herein.

Embodiment 22. The method or adhesive composition of any of the preceding Embodiments, wherein the adhesive composition comprises, consists essentially of, or consists of a polymeric composition, such as a crosslinked polymeric composition.

Embodiment 23. The method or adhesive composition of any of the preceding Embodiments, wherein the adhesive composition comprises, consists essentially of, or consists of (i) one or more hydrophilic polymers, one or more zwitterionic polymers, or a combination thereof; (ii) one or more tissue coupling groups; (iii) one or more crosslinkers; or (iv) a combination thereof.

Embodiment 24. The method or adhesive composition of any of the preceding Embodiments, wherein the one or more hydrophilic polymers comprise, consist essentially of, or consist of polyethylene glycol, polyacrylic acid, polyacrylamide, polyvinyl alcohol, polyhydroxy ethyl methacrylate, polyethylene glycol, polyurethane, casein, albumin, gelatin, chitosan, dextran, hyaluronic acid, alginate, cellulose, polyvinyl pyrrolidone, polystyrene sulfonate, collagen, alginic acid, pectin, or a combination thereof.

Embodiment 25. The method or adhesive composition of Embodiment 24,wherein the alginate and the cellulose are oxidized alginate and oxidized cellulose, respectively.

Embodiment 26. The method of adhesive composition of any of the preceding Embodiments, wherein the one or more zwitterionic polymers comprise, consist essentially of, or consist of poly(phospobetaine), poly(carboxybetaine), poly(sulfobetaine), or a combination thereof.

Embodiment 27. The method or adhesive composition of any of the preceding Embodiments, wherein the one or more tissue coupling groups comprise, consist essentially of, or consist of an amine coupling group, a thiol coupling group, a cysteine coupling group, an N-acetyl-cysteine coupling group, a boronate ester coupling group, or a combination thereof. Embodiment 28. The method or adhesive composition of any of the preceding

Embodiments, wherein the amine coupling group comprises, consists essentially of, or consists of an N-hydroxysuccinimide ester (PAAc-co-NHS ester), a PAAm-co-NHS, an N-Hydroxysuccinimide (NHS) PEG, a poly(L-lactide-co-glycolide)-NHS, a poly(D,L-lactide)-polyethylene glycol-CO-NHS, a poly(N-isopropylacrylamide) N-hydroxysuccinimide terminated, an aldehyde, an imidoester, an epoxide, an isocyanate, a catechol, and a combination thereof.

Embodiment 29. The method or adhesive composition of any of the preceding Embodiments, wherein the thiol coupling group comprises, consists essentially of, or consists of alginate, albumin, fibrinogen, collagen, chitosan, gelatin, or a combination thereof.

Embodiment 30. The method or adhesive composition of any of the preceding Embodiments, wherein the cysteine coupling group comprises, consists essentially of, or consists of fibrinogen, collagen, or a combination thereof.

Embodiment 31. The method or adhesive composition of any of the preceding Embodiments, wherein the one or more boronate ester coupling groups comprise, consist essentially of, or consist of acrylamide, N-isopropylacrylamide, polyvinyl alcohol (PVA), alginate, cellulose, and a combination thereof.

Embodiment 32. The method or adhesive composition of any of the preceding Embodiments, wherein the one or more crosslinkers comprise, consist essentially of, or consist of gelatin methacrylate, hyaluronic acid methacrylate, oxidized methacrylic alginate, polycaprolactone diacrylate, N,N′-bis(acryloyl) cystamine, N,N′-methylenebis(acrylamide), polyethylene glycol diacrylate, polyethylene glycol dimethacrylate, or a combination thereof.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1—Preparation and Testing of Adhesive Composition

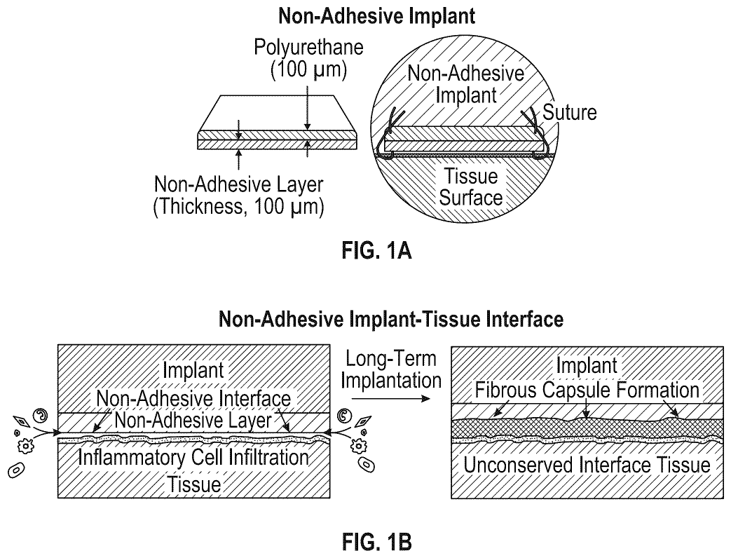

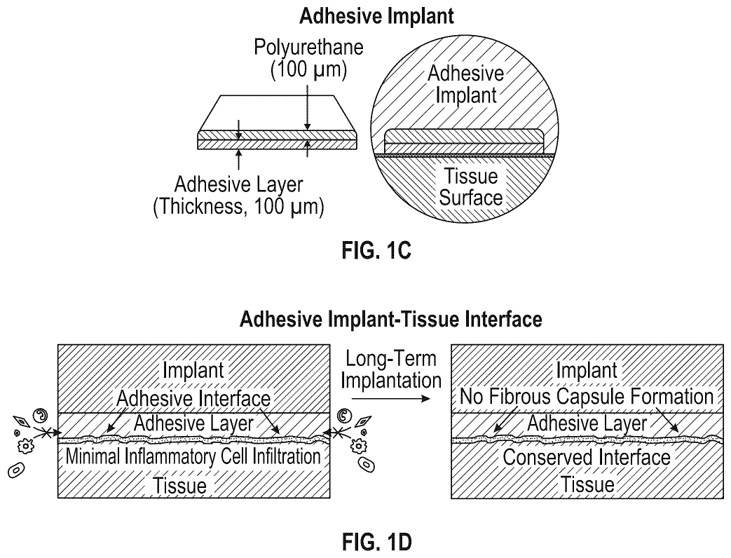

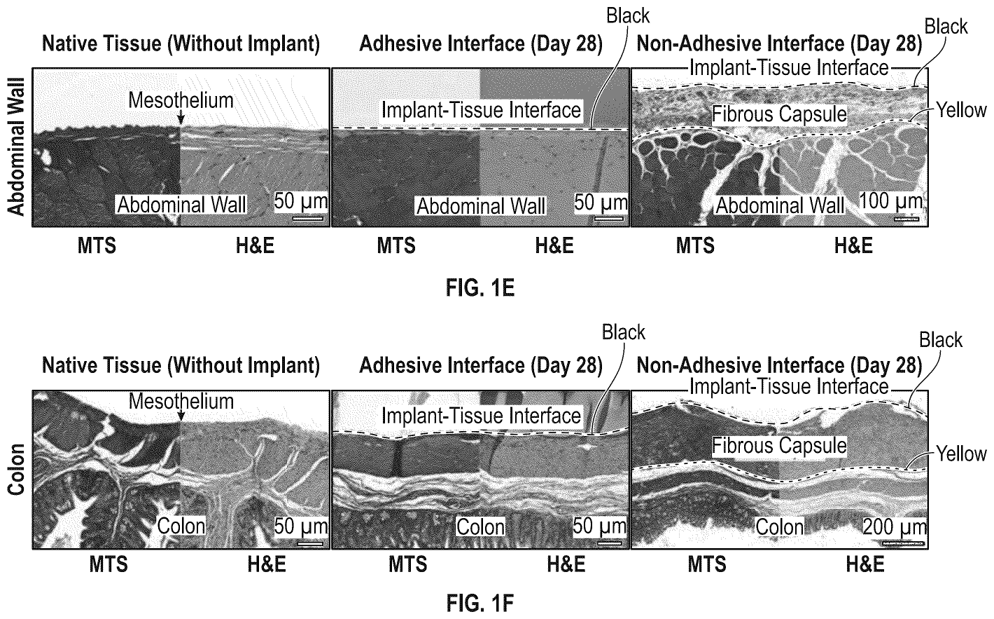

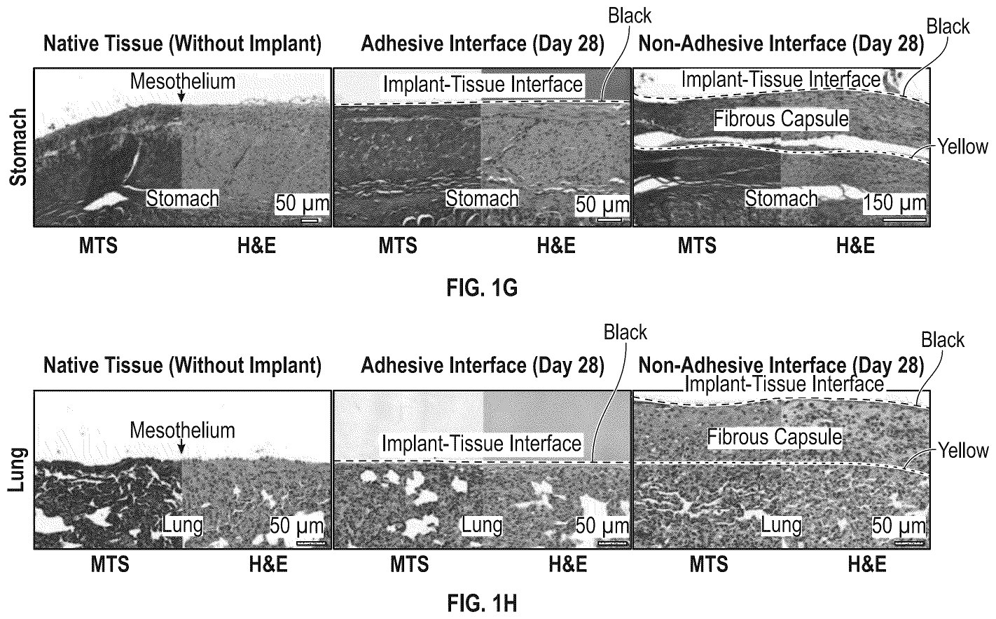

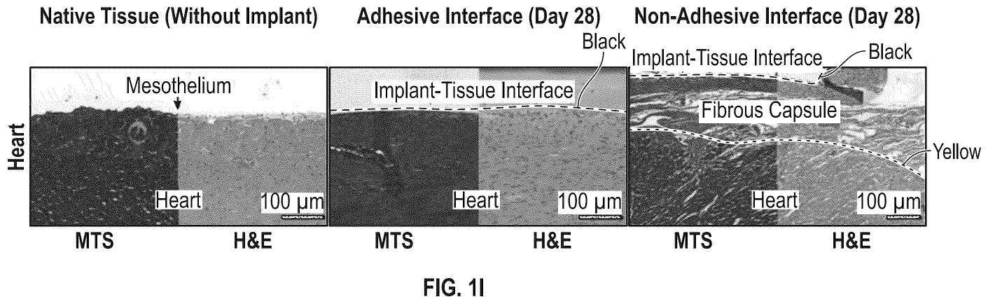

This example demonstrated that an embodiment of an adhesive implant described herein provided mechanical integration of the implant to the target tissue, and also avoided fibrous capsule formation at the implant-tissue interface (FIG. 1C,1D).

It was hypothesized that the conformal interfacial integration between the adhesive implant and the tissue surface would minimize the establishment of the inflammatory microenvironment (e.g., protein adsorption) and subsequent infiltration of inflammatory cells (e.g., neutrophils, monocytes, macrophages) at the implant-tissue interface, and avoid the collagen deposition and fibrous capsule formation in the long-term (FIG. 1D). In contrast, conventional non-adhesive implants did not form conformal integration with the tissue surface, which allowed the infiltration of inflammatory cells to the implant-tissue interface and subsequent formation of the fibrous capsule (FIG. 1B).

In this example, an adhesive implant was prepared that included a mock device (polyurethane), and an adhesive layer including interpenetrating networks between the covalently-crosslinked poly(acrylic acid) NHS ester and physically-crosslinked poly(vinyl alcohol) (FIG. 1C) (Yuk, H. et al. Nature 575, 169-174 (2019); Wu, J. et al. Science Translational Medicine 14, eabh2857 (2022)). The adhesive layer provided highly conformal and stable integration of the implant to wet tissues (Deng, J. et al., Nature Materials 20, 229-236 (2021). Also prepared was a non-adhesive implant by fully swelling the same mock device and adhesive layer in a phosphate-buffered saline (PBS) bath before implantation to remove the adhesive property (Chen, X., et al., Proceedings of the National Academy of Sciences 117, 15497-15503 (2020)), while keeping the chemical compositions of the implant identical (FIG. 1A).

FIG. 1A and FIG. 1B are schematic illustrations of a non-adhesive implant consisting of a mock device (polyurethane) and non-adhesive layer (FIG. 1A) and long-term in vivo implantation with fibrous capsule formation at the implant-tissue interface (FIG. 1B). FIG. 1C and FIG. 1D are schematic illustrations of an adhesive implant consisting of the mock device (polyurethane) and adhesive layer (FIG. 1C) and long-term in vivo implantation without fibrous capsule formation at the implant-tissue interface (FIG. 1D). FIG. 1E-FIG. 1I are representative histology images stained with Masson's trichrome (MTS) and hematoxylin and eosin stain (H&E) for native tissue (left), the adhesive implant (middle), and non-adhesive implant (right) collected on day 28 post-implantation to the abdominal wall (FIG. 1E), colon (FIG. 1F), stomach (FIG. 1G), lung (FIG. 1H), and heart (FIG. 1I). Black and yellow dotted lines in the images indicate the implant-tissue interface and the mesothelium-fibrous capsule interface, respectively.

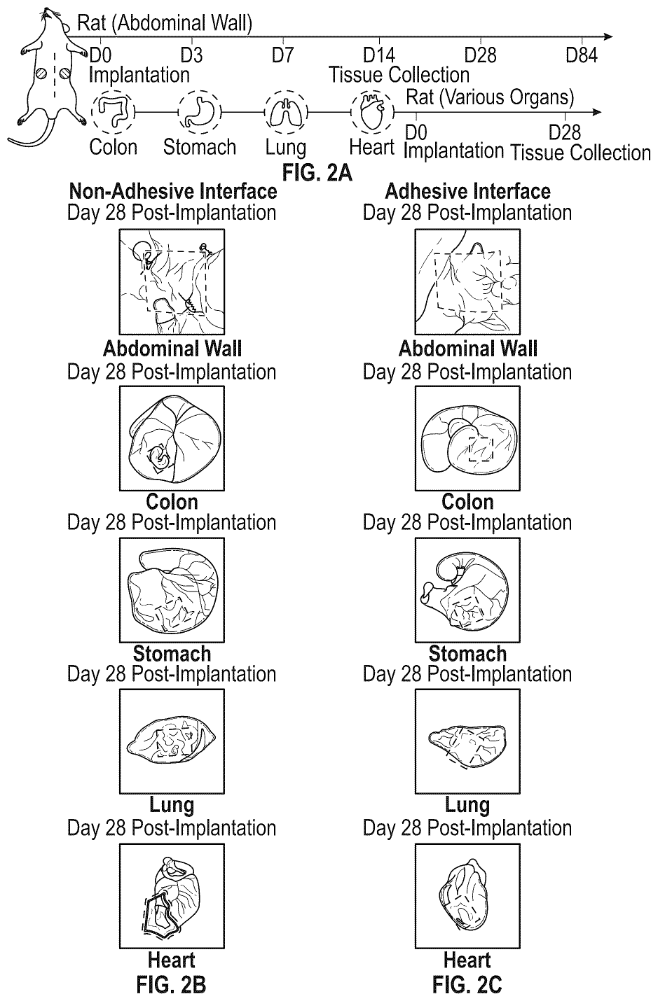

Both adhesive and non-adhesive implants were implanted on the surfaces of various organs including the abdominal wall, colon, stomach, lung, and heart by using rat models in vivo for up to 84 days (FIG. 2A). The non-adhesive implant was sutured on the organ surfaces. Macroscopic observations indicated that the adhesive and non-adhesive implants stably remained at the implantation site on the organ surfaces (FIG. 2B,2C). Specifically, FIG. 2A includes schematic illustrations for in vivo rat studies. FIG. 2B and FIG. 2C are photographs of various organs collected on day 28 post-implantation for the non-adhesive implant (FIG. 2B) and the adhesive implant (FIG. 2C). Black dotted lines in photographs indicate the boundary of implants.

To analyze the foreign body reaction and fibrous capsule formation for the adhesive and non-adhesive implants, histological analysis was performed of the native tissue, adhesive implant, and non-adhesive implant for various organs (FIG. 1E-1I).

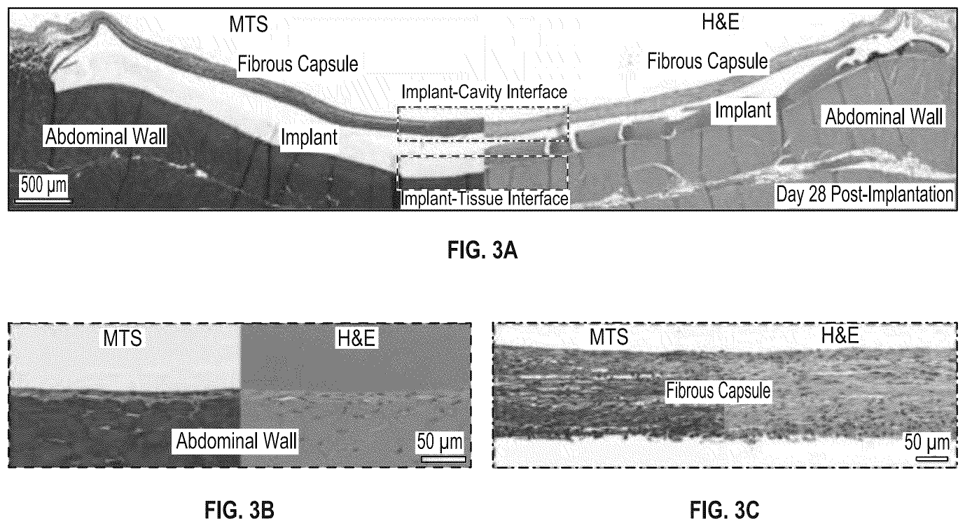

Histological evaluation by a blinded pathologist indicated that the adhesive implant formed conformal integration with the organ surface (i.e., mesothelium), and prevented the formation of the fibrous capsule on day 28 post-implantation for all organs (abdominal wall, colon, stomach, lung, heart) (FIGS. 1E-1I and 3A,3B). FIG. 3A includes representative histology images stained with Masson's trichrome (left) and hematoxylin and eosin stain (H&E, right) of the adhesive implant collected on day 28 post-implantation to the abdominal wall. Black and red dotted areas indicate the implant-tissue interface and the implant-abdominal cavity interface, respectively. FIG. 3B and FIG. 3C are representative histology images stained with Masson's trichrome (left) and H&E (right) of the implant-tissue interface (FIG. 3B) and implant-cavity interface (FIG. 3C) for the adhesive implant collected on day 28 post-implantation to the abdominal wall

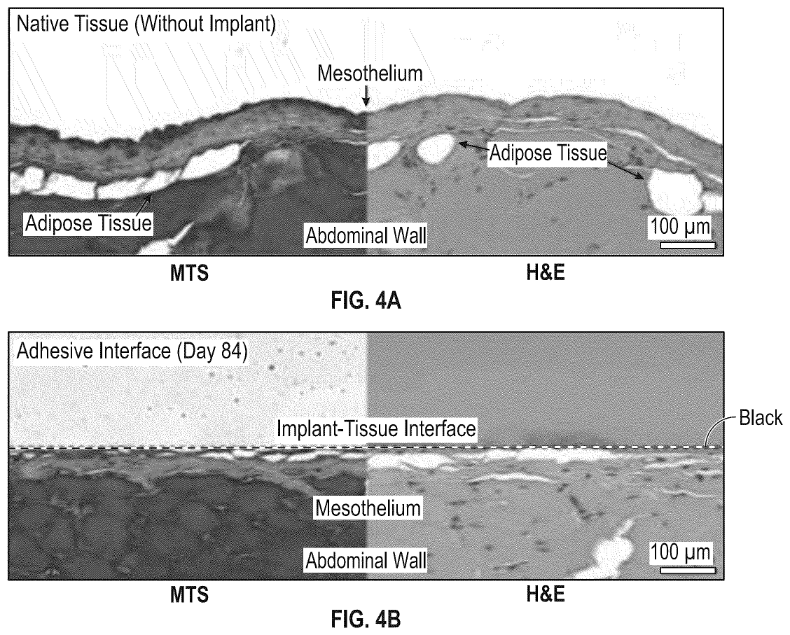

Notably, the fibrous capsule-free adhesive implant-tissue interface was maintained over 84 days post-implantation (FIG. 4A and FIG. 4B). FIG. 4A includes representative histology images stained with Masson's trichrome (MTS, left) and hematoxylin and eosin stain (H&E, right) of the native abdominal wall tissue without implant collected on day 84 post-surgery. FIG. 4B includes representative histology images stained with MTS (left) and H&E (right) of the adhesive implant collected on day 84 post-implantation to the abdominal wall. Black dotted lines in the images indicate the implant-tissue interface.

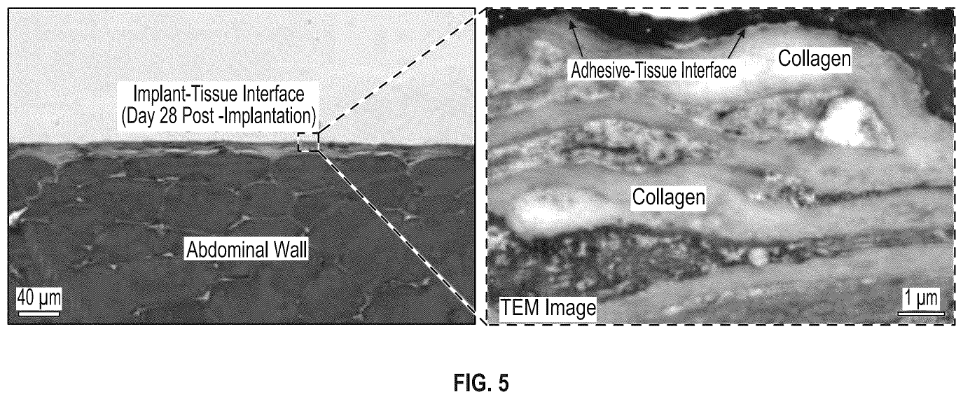

Furthermore, a transmission electron microscope (TEM) image of the adhesive implant-tissue interface showed that the adhesive layer maintained highly conformal and gapless integration with the collagenous layer of the mesothelium on a subcellular scale on day 28 post-implantation (FIG. 5). FIG. 5 is a representative histology image stained with Masson's trichrome (left) and TEM image (right) of the adhesive implant collected on day 28 post-implantation to abdominal wall. * in images indicates the implant.

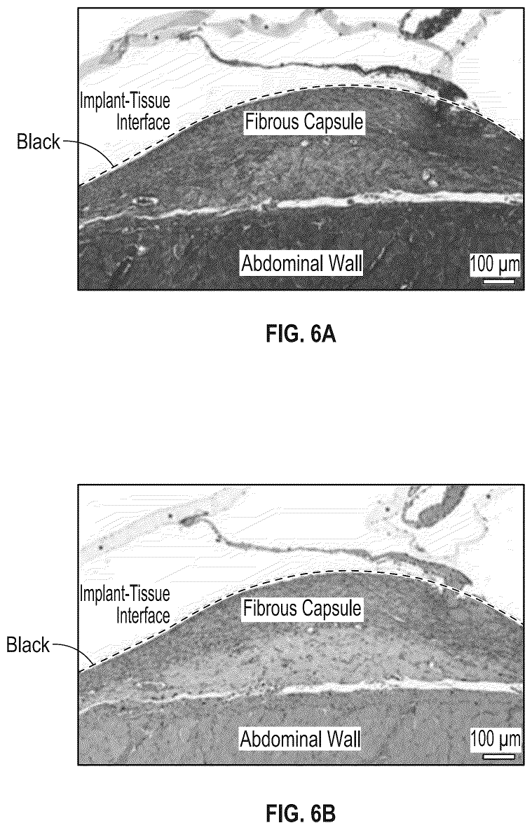

In contrast, the non-adhesive implant caused substantial formation of the fibrous capsule at the implant-tissue interface for all organs on day 28 post-implantation, which was consistent with the foreign body reaction to the mock device alone (FIG. 6A and FIG. 6B). FIG. 6A and FIG. 6B include representative histology images stained with Masson's trichrome (FIG. 6A) and hematoxylin and eosin stain (FIG. 6B) of the polyurethane mock device collected on day 28 post-implantation to the abdominal wall. The “*” in the images indicates the implant; black dotted lines indicate the implant-tissue interface.

Unlike the adhesive-tissue interface, the mock device-cavity interface of the adhesive implant resulted in fibrous capsule formation similar to the non-adhesive implant (FIG. 3A,3C), which further confirmed that the adhesive interface was required to prevent fibrous capsule formation.

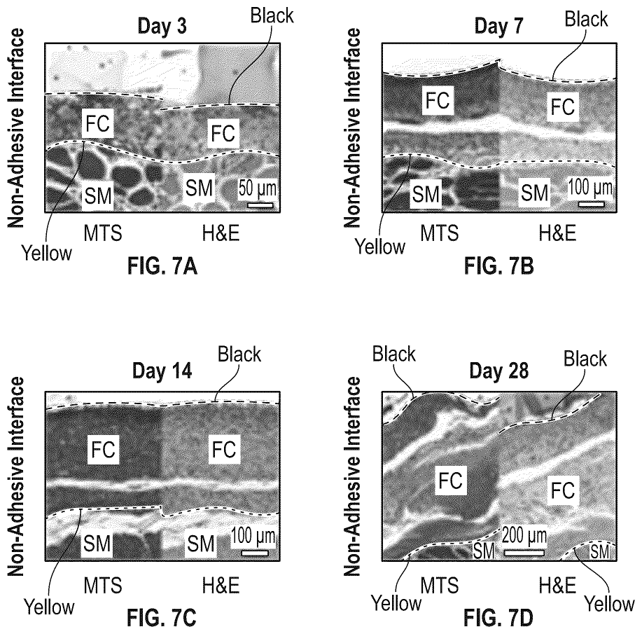

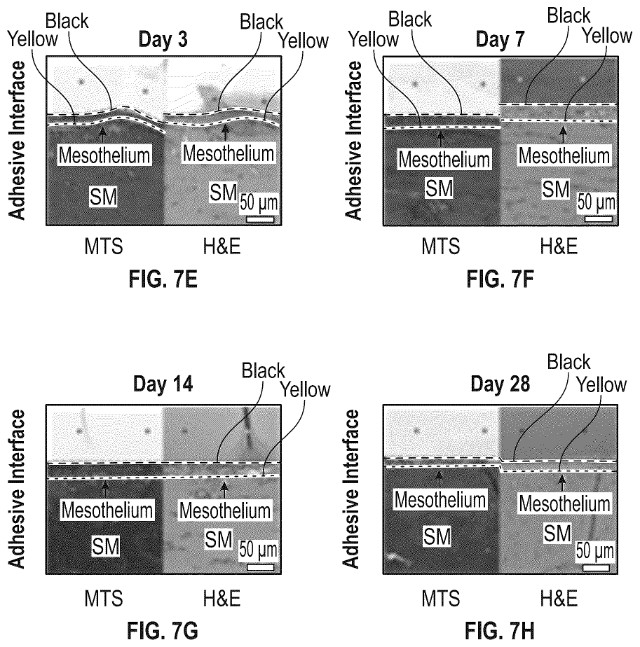

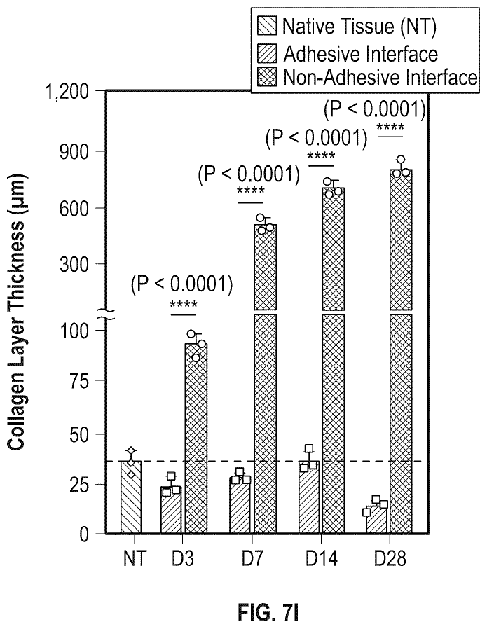

To assess the foreign body reaction and fibrous capsule formation over time, histological analyses were conducted for the adhesive and non-adhesive implants to the abdominal wall on day 3, day 7, day 14, and day 28 post-implantation (FIG. 7A-7H). The collagen layer thickness at the implant-tissue interface remained comparable to the native tissue (i.e., the thickness of mesothelium) for the adhesive implant at all time points (FIG. 7I). In contrast, the collagen layer thickness at the non-adhesive implant-tissue interface increased over time due to the formation of fibrous capsule, and was significantly thicker than both native tissue and adhesive implant at all time points (FIG. 7I).

FIG. 7A-FIG. 7D are representative histology images stained with Masson's trichrome (MTS, left) and hematoxylin and eosin (H&E, right) of the non-adhesive implant collected on day 3 (FIG. 7A), day 7 (FIG. 7B), day 14 (FIG. 7C), and day 28 (FIG. 7D) post-implantation to the abdominal wall. FIG. 7E-FIG. 7H are representative histology images stained with MTS (left) and H&E (right) of the adhesive implant collected on day 3 (FIG. 7E), day 7 (FIG. 7F), day 14 (FIG. 7G), and day 28 (FIG. 7H) post-implantation to the abdominal wall. * in images indicates the implant; black dotted lines in images indicate the implant-tissue interface; yellow dotted lines in images indicate the mesothelium-fibrous capsule (non-adhesive implant) or the mesothelium-skeletal muscle (adhesive implant) interface. SM, skeletal muscle; FC, fibrous capsule. FIG. 7I depicts collagen layer thickness at the implant-tissue interface measured at different time points post-implantation. The Blue dotted line indicates the average collagen layer thickness of the native tissue. Values represent the mean and the standard deviation (n=3; independent samples). Statistical significance and P values are determined by two-sided unpaired 1-tests; ****P<0.0001.

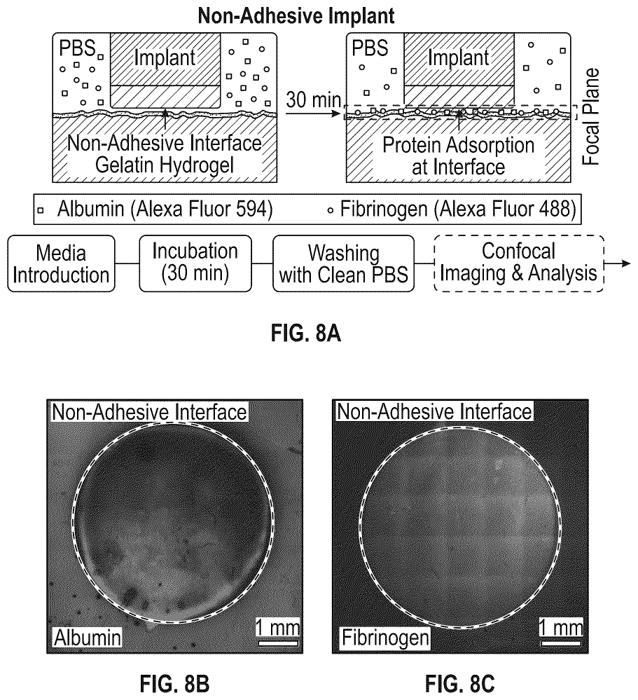

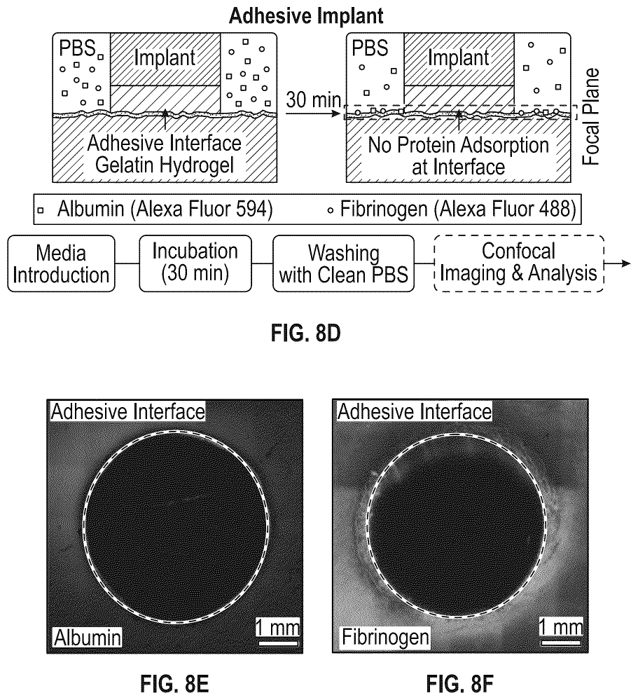

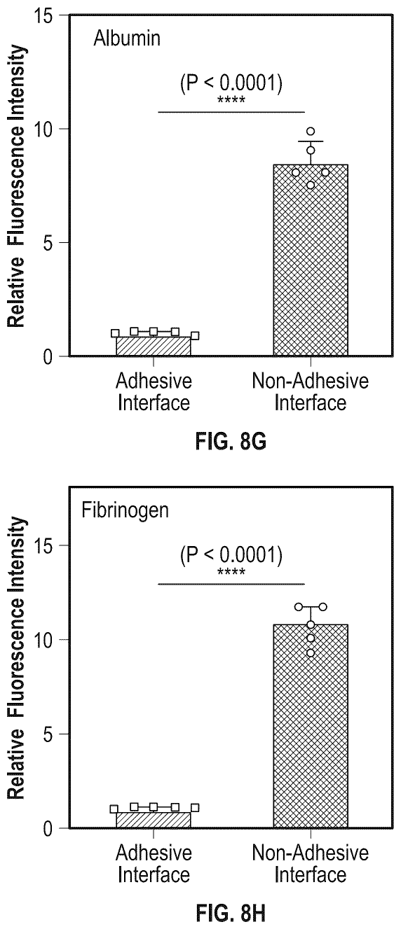

To further investigate the hypothesis of this example, a set of characterizations was performed for key participants of the foreign body reaction, including in vitro protein adsorption assay, immunofluorescence analysis, Luminex quantification, quantitative PCR (qPCR), and RNA sequencing analysis. A protein adsorption assay with fluorescently-labeled albumin and fibrinogen was performed to evaluate the adhesion of proteins at the implant-tissue interface during the initial stage of the foreign body reaction (FIG. 8) (Swartzlander, M. D. et al. Biomaterials 41, 26-36 (2015); Hedayati, M. et al., Acta Biomaterialia 102, 169-180 (2020)). After a 30 minute co-culture in the protein solution, the adhesive implant-substrate interface showed significantly lower protein adsorption compared to the non-adhesive implant-substrate interface (P<0.0001) for both fluorescently-labeled albumin and fibrinogen (FIG. 8G,8H), which demonstrated the adhesive interface's capability to prevent non-specific protein adsorption.

FIG. 8A-FIG. 8C include a schematic illustration of the experimental setup (FIG. 8A), and representative confocal microscope images for in vitro albumin (FIG. 8B) and fibrinogen (FIG. 8C) adsorption assay for the non-adhesive implant. FIG. 8D-FIG. 8F include a schematic illustration of the experimental setup (FIG. 8D) and representative confocal microscope images for in vitro albumin (FIG. 8E) and fibrinogen (FIG. 8F) adsorption assay for the adhesive implant. FIG. 8G and FIG. 8H depict a relative fluorescence intensity at the implant-substrate interface measured 30 min after co-culture for albumin (FIG. 8G) and fibrinogen (FIG. 8H). Values in FIG. 8G and FIG. 8H represent the mean and the standard deviation (n=5). Statistical significance and P values are determined by two-sided unpaired t-tests; ****P<0.0001.

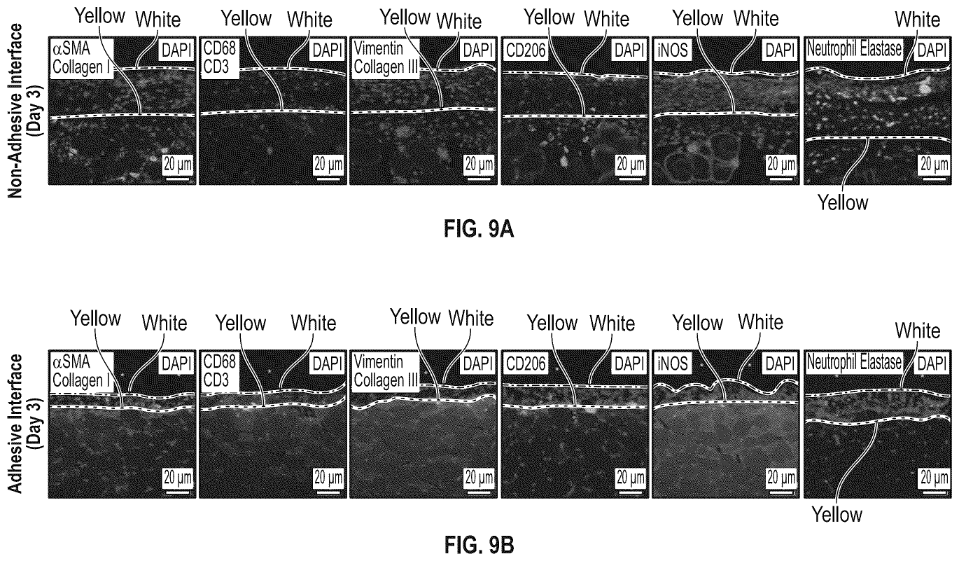

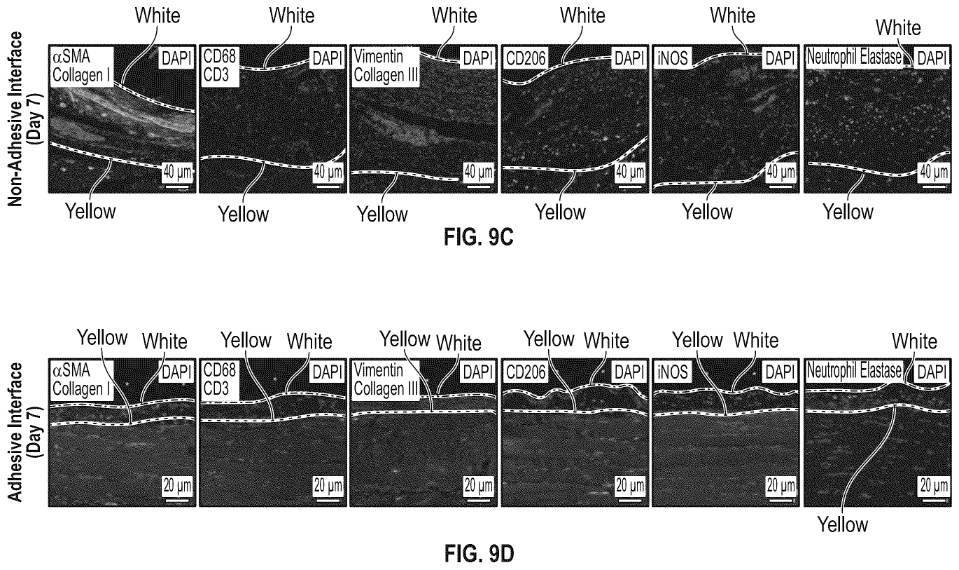

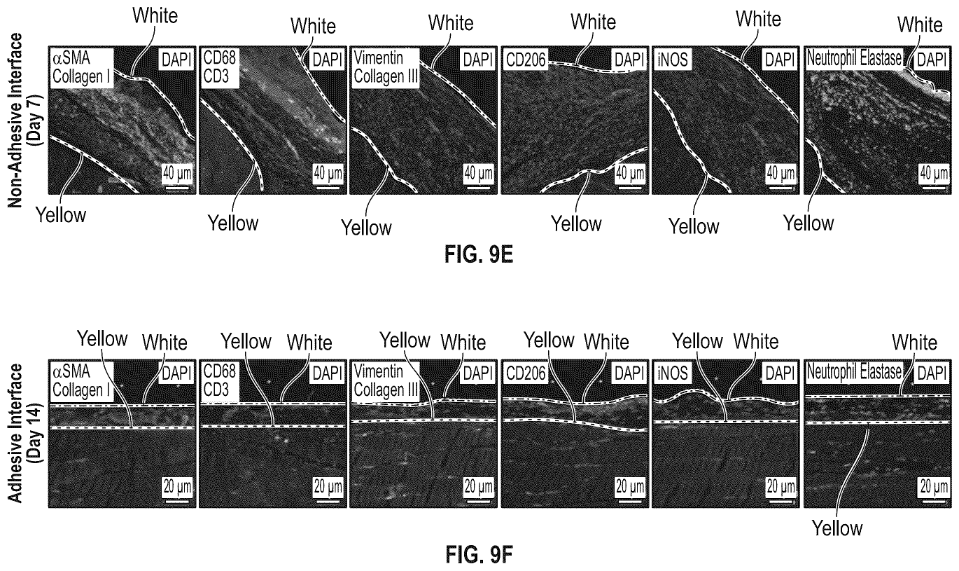

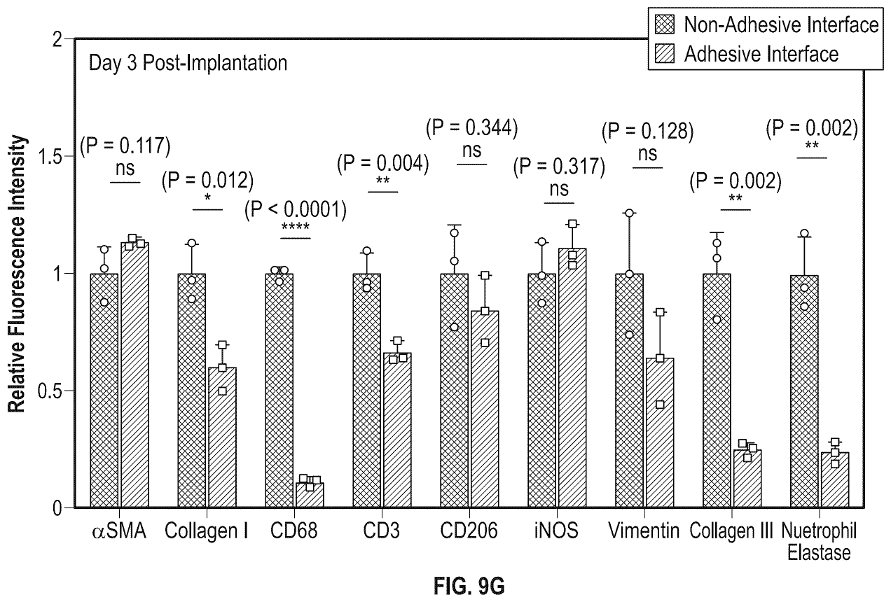

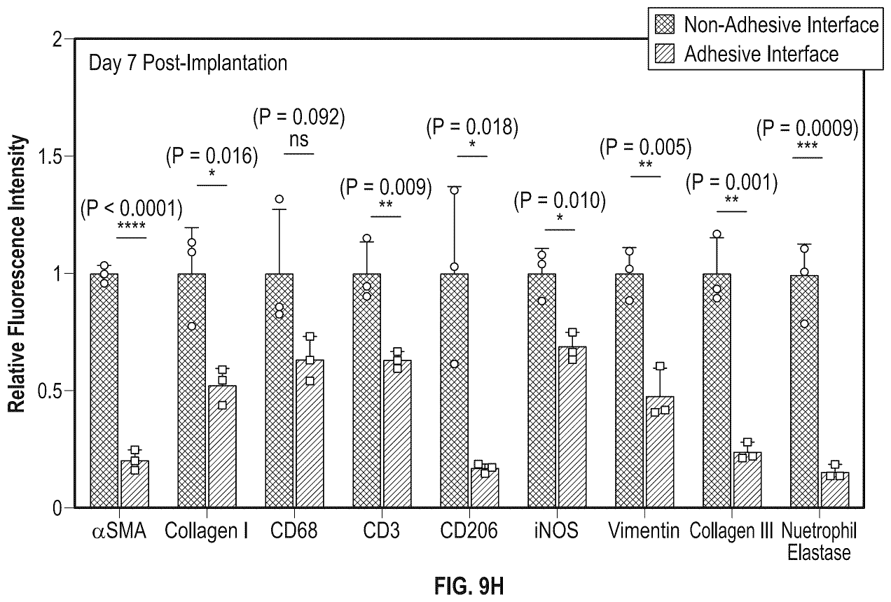

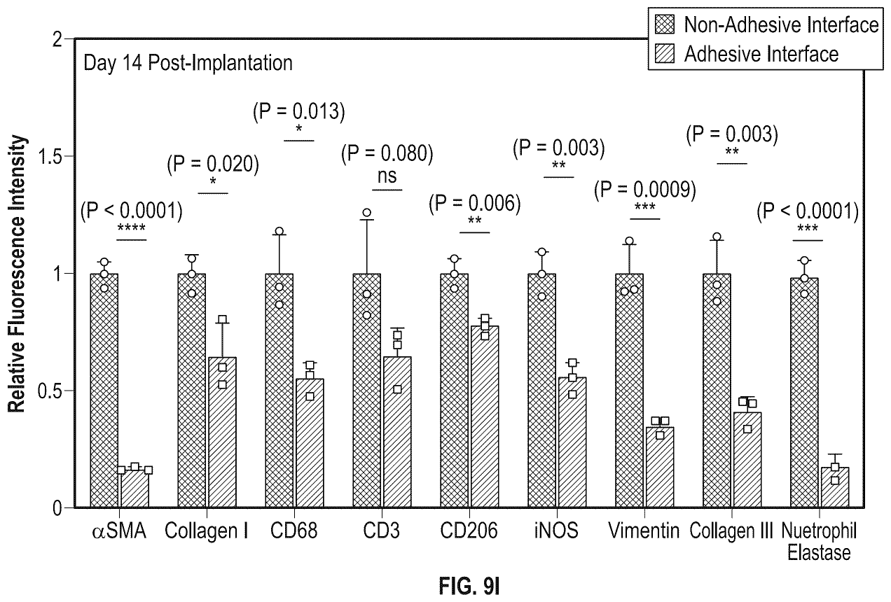

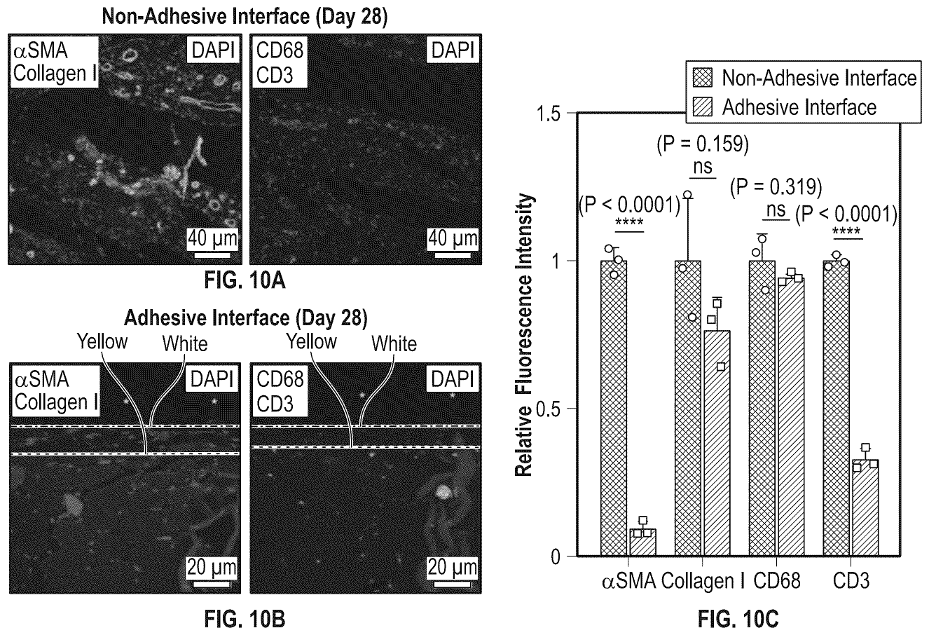

To investigate the infiltration of immune cells and subsequent fibrous capsule formation at the implant-tissue interface, immunofluorescence staining and normalized immunofluorescence intensity analysis were performed (FIG. 9 and FIG. 10) for fibroblasts (αSMA, Collagen III), neutrophils (Neutrophil elastase), macrophages (CD68 for pan-macrophage; iNOS and Vimentin for pro-inflammatory macrophage; CD206 for anti-inflammatory macrophage), T-cells (CD3), and fibrosis (Collagen I). The normalized fluorescence intensity analysis showed that the adhesive implant-tissue interface exhibited significantly less expression for fibroblasts (αSMA, Collagen III), neutrophils (Neutrophil elastase), and fibrosis (Collagen I) compared to the non-adhesive implant-tissue interface at all time points (P≤0.01) except on day 3 post-implantation for αSMA expression (P=0.117) (FIG. 9G-FIG. 9I).

FIG. 9A, FIG. 9C, and FIG. 9E include representative immunofluorescence images of the non-adhesive implant collected on day 3 (FIG. 9A), day 7 (FIG. 9C), and day 14 (FIG. 9F) post-implantation to the abdominal wall. FIG. 9B, FIG. 9D, and FIG. 9F include representative immunofluorescence images of the adhesive implant collected on day 3 (FIG. 9B), day 7 (FIG. 9D), and day 14 (FIG. 9F) post-implantation to the abdominal wall. In immunofluorescence images, cell nuclei are stained with 4′,6-diamidino-2-phenylindole (DAPI, blue); green fluorescence corresponds to the expression of fibroblast (αSMA), Vimentin, neutrophil (Neutrophil Elastase), and macrophage (CD68, CD206, iNOS); red fluorescence corresponds to the expression of type 1 collagen (Collagen I), type 3 collagen (Collagen III), and T cell (CD3). * in images indicates the implant; white dotted lines in images indicate the implant-tissue interface; yellow dotted lines in images indicate the mesothelium-fibrous capsule (non-adhesive implant) or the mesothelium-skeletal muscle (adhesive implant) interface. FIG. 9G-FIG. 9I depict normalized fluorescence intensity from the immunofluorescence images at different time points for day 3 (FIG. 9G), 7 (FIG. 9H), and 14 (FIG. 9I) post-implantation. Values in FIG. 9G-FIG. 9I represent the mean and the standard deviation (n=3; independent samples). Statistical significance and P values are determined by two-sided unpaired t-tests; ns, not significant; *P<0.05; **P≤0.01; ***P≤0.001; ****P<0.0001.

Furthermore, the adhesive implant-tissue interface showed lower expression for immune cells, such as neutrophils (Neutrophil elastase+), macrophages (CD68+, iNOS+, Vimentin+, CD206+), and T-cells (CD3+) compared to the non-adhesive implant-tissue interface at all time points (FIG. 9G-FIG. 9I), except on day 3 post-implantation for iNOS expression (P=0.317).

FIG. 10A includes representative immunofluorescence images of the non-adhesive implant collected on day 28 post-implantation to the abdominal wall. FIG. 10B includes representative immunofluorescence images of the adhesive implant collected on day 28 post-implantation to the abdominal wall. In immunofluorescence images, cell nuclei are stained with 4′,6-diamidino-2-phenylindole (DAPI, blue); green fluorescence corresponds to the expression of fibroblast (αSMA) and macrophage (CD68); red fluorescence corresponds to the expression of type 1 collagen (Collagen I) and T cell (CD3). The “*” in the images indicates the implant; white dotted lines in images indicate the implant-tissue interface; yellow dotted lines in images indicate the mesothelium-skeletal muscle interface. FIG. 10C depicts normalized fluorescence intensity from the immunofluorescence images for day 28 post-implantation. Values in FIG. 10C represent the mean and the standard deviation (n=3; independent samples). Statistical significance and P values are determined by two-sided unpaired t-tests; ns, not significant; ****P<0.0001.

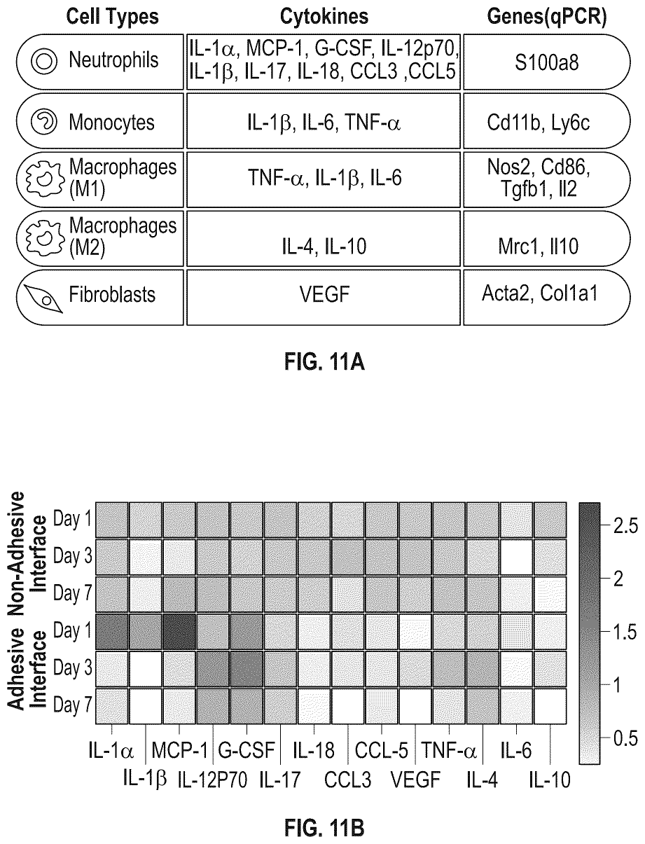

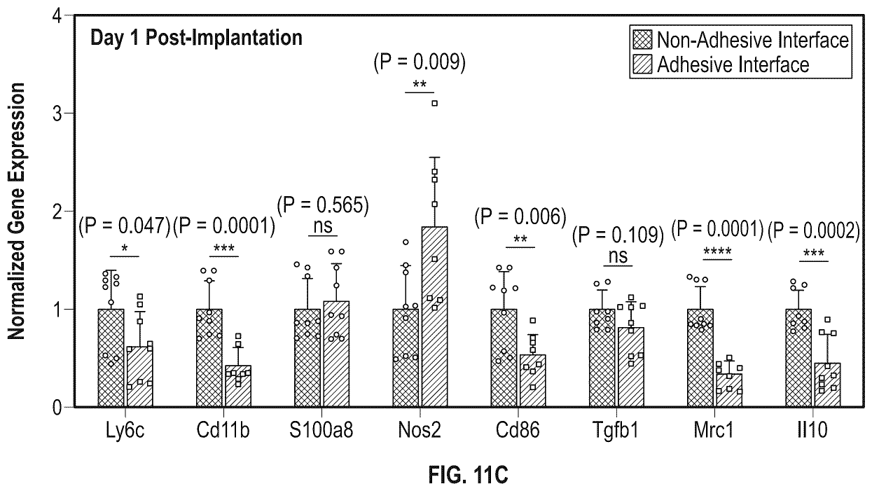

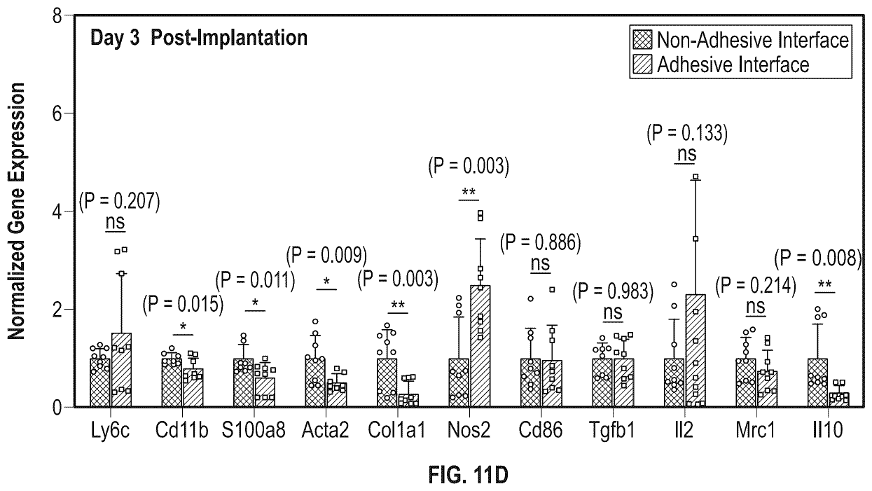

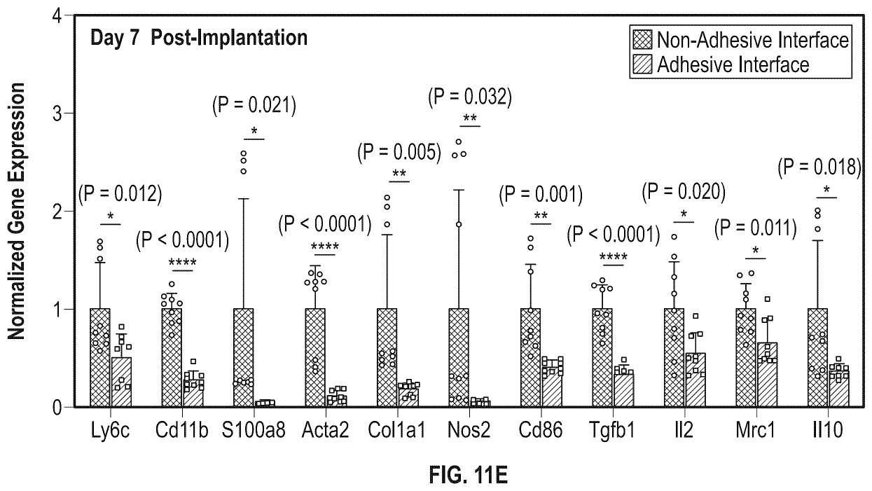

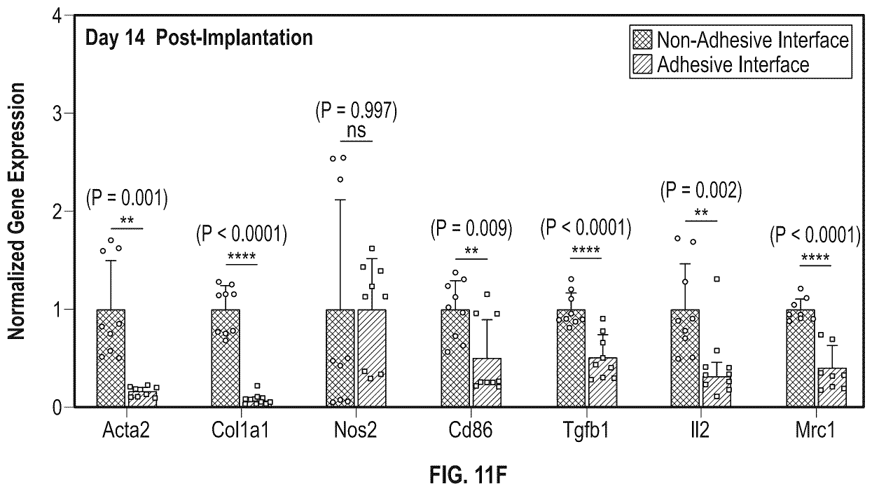

To further delineate the immune response at the implant-tissue interface, immune cell-related cytokines/chemokines and genes were profiled by using Luminex quantification and qPCR analysis, respectively (FIG. 11). A multiplex protein assay on day 1 post-implantation to the abdominal wall for adhesive implant-tissue interface exhibited an increase trend in several cytokines (IL-1α, IL-1β, MCP-1), which subsequently became a lower trend on days 3 and 7 post-implantations compared to the non-adhesive implant-tissue interface (FIG. 11B), which suggested the presence of distinct immune populations at the very early time point. Notably, the adhesive implant-tissue interface on days 1, 3, and 7 post-implantation showed lower amounts of various pro-inflammatory cytokines and chemokines (IL-18, CCL3, CCL5) compared to the non-adhesive implant-tissue interface at all time points evaluated (FIG. 11B). Inflammatory regulator cytokines, such as G-CSF and IL-12p70, showed increased expression for the adhesive implant-tissue interface on days 3 and 7 post-implantations compared to the non-adhesive implant-tissue interface, which are known to inhibit pro-inflammatory cytokine secretion during the acute stage of the foreign body reaction (Rutella, S. et al., The Journal of Immunology 175, 7085-7091 (2005)).

qPCR analysis showed no difference in neutrophil marker S100a8 expression on day 1, but a significant decrease for subsequent time points (days 3 and 7) for the adhesive implant-tissue interface, which was in line with collected immunofluorescence data. This suggested that even though the initial recruitment of neutrophils was similar, possibly due to the performed surgery, it was followed by rapid neutrophil clearance in the case of the adhesive implant-tissue interface and not by prolonged presence as seen in the non-adhesive implant.

Moreover, Nos2 exhibited a significantly higher expression for the adhesive implant-tissue interface compared to the non-adhesive implant-tissue interface (P≤0.01) on days 1 and 3 post-implantation (FIG. 11C-FIG. 11E). This higher expression of Nos2 was in agreement with the increased levels of the inflammatory regulator cytokines (G-CSF, IL-12p70) during the acute time points (FIG. 11B), which were previously reported to mediate the inflammatory response via activating iNOS or inhibiting the secretion of pro-inflammatory cytokines (Cassini-Vieira, P. et al., Mediators of Inflammation 2015, 138461 (2015)).

FIG. 11A depicts cytokines and genes relevant to each cell type in the Luminex and q-PCR studies. FIG. 11B is a heatmap of immune response-related cytokines and chemokines measured with Luminex assay of the non-adhesive and the adhesive implant-tissue interface collected on days 1, 3, and 7 post-implantations to the abdominal wall. FIG. 11C-FIG. 11F depict normalized gene expression of immune response-related markers for the non-adhesive and the adhesive implant-tissue interface collected on day 1 (FIG. 11C), day 3 (FIG. 11D), day 7 (FIG. 11E), and day 14 (FIG. 11F) post-implantations to the abdominal wall. Values in FIG. 11C-FIG. 11F represent the mean and the standard deviation (n=9; three samples per animal). Statistical significance and P values are determined by two-sided unpaired t-tests; ns, not significant; *P<0.05; **P≤0.01; ***P≤0.001; ****P<0.0001.

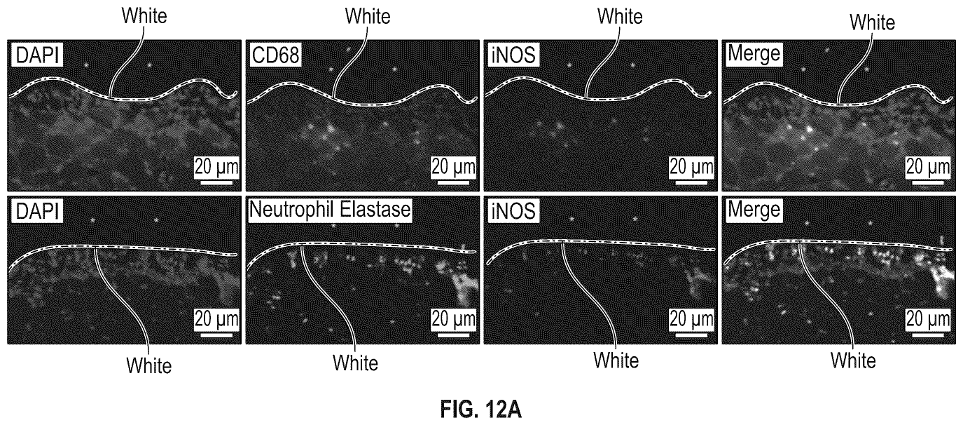

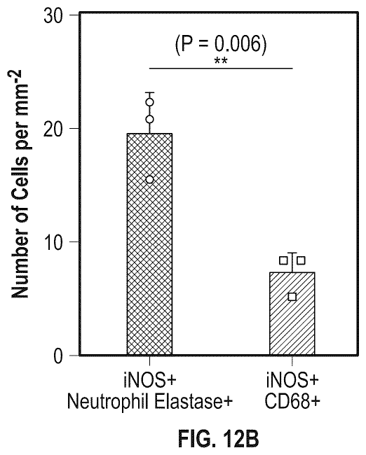

To further investigate the source of Nos2 during the acute time points (days 1 and 3 post-implantation), double immunofluorescence staining was performed for iNOS/Neutrophil elastase and iNOS/CD68 (FIG. 12A). The immunofluorescence staining showed that there was a higher density of iNOS+neutrophils rather than macrophages on day 3 post-implantation, which indicated that the adhesive implant-tissue interface favored an iNOS producing neutrophil subset (FIG. 12B) (see, e.g., Saini, R. et al. Journal of Leukocyte Biology 79, 519-528 (2006)).

FIG. 12A includes representative immunofluorescence images of the adhesive implant collected on day 3 post-implantation to the abdominal wall. In immunofluorescence images, cell nuclei are stained with 4′,6-diamidino-2-phenylindole (DAPI, blue); green fluorescence corresponds to the expression of macrophage (CD68) and neutrophil (Neutrophil Elastase); red fluorescence corresponds to the expression of iNOS. * in images indicates the implant; white dotted lines in images indicate the implant-tissue interface. FIG. 12B depicts quantification of iNOS+/Neutrophil Elastase+ and iNOS+/CD68+ cells per unit area on day 3 post-implantation. Values in FIG. 12B represent the mean and the standard deviation (n=3; independent samples). Statistical significance and P values are determined by two-sided unpaired t-tests; **P≤0.01.

On day 7 post-implantation, immune cell-related genes (S100a8 for neutrophils; Ly6c, Cd11b for monocytes; Nos2, Cd86, Tgfb1 for pro-inflammatory macrophages; Mrc1, Il10 for anti-inflammatory macrophages; Il2 for T-cells) exhibited a significantly lower expression for the adhesive implant-tissue interface compared to the non-adhesive implant-tissue interface (P<0.05) (FIG. 11E). On day 14 post-implantation, immune response-related genes (Cd86, Mrc1, Tgfb1, Il2) still exhibited a significantly lower expression for the adhesive implant-tissue interface compared to the non-adhesive implant-tissue interface (P≤0.01) (FIG. 11F). Fibrosis-related genes (Colla1, Acta2) had a significantly lower expression for the adhesive implant-tissue interface compared to the non-adhesive implant-tissue interface (P≤0.01) on days 3, 7, and 14 post-implantations (FIG. 11D-FIG. 11F).

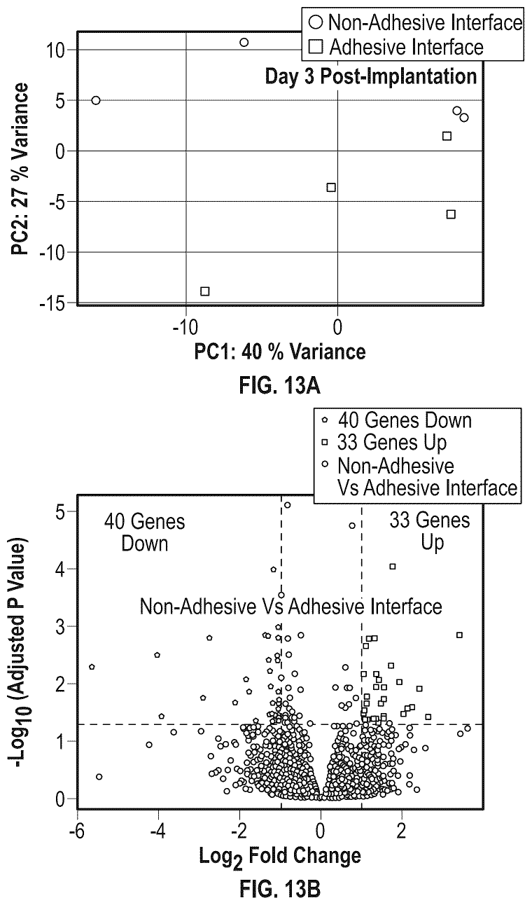

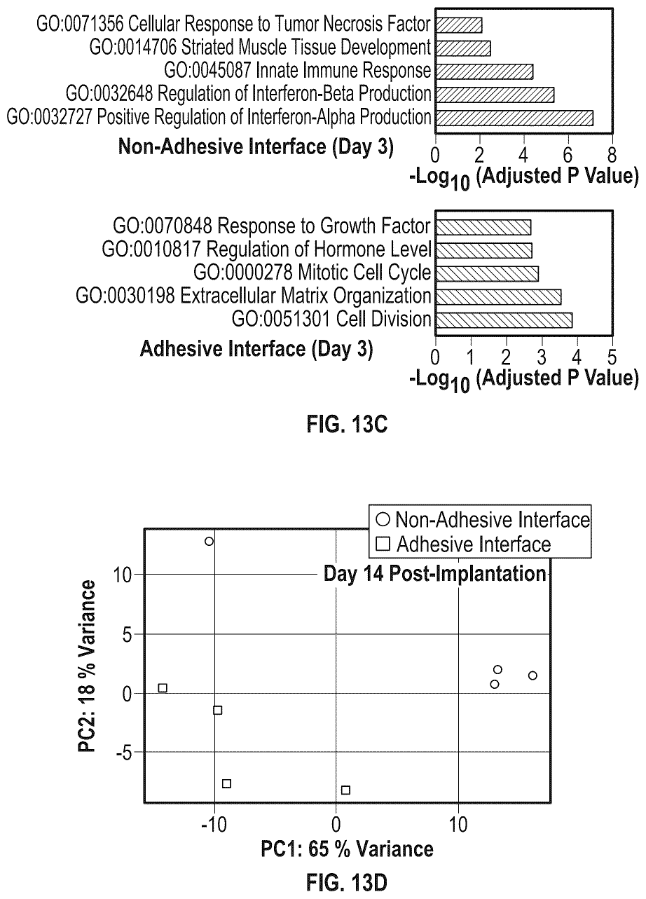

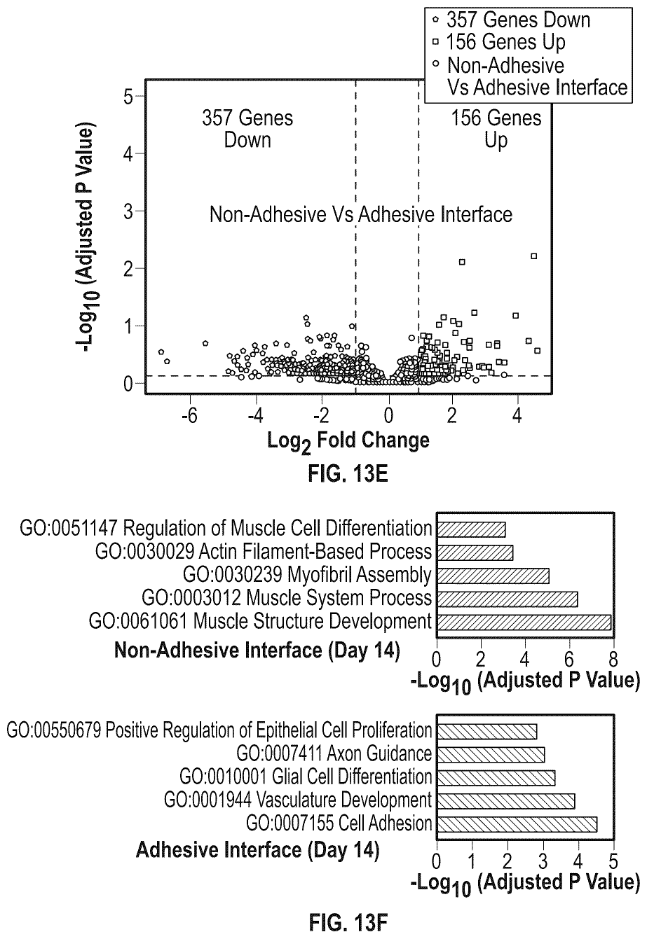

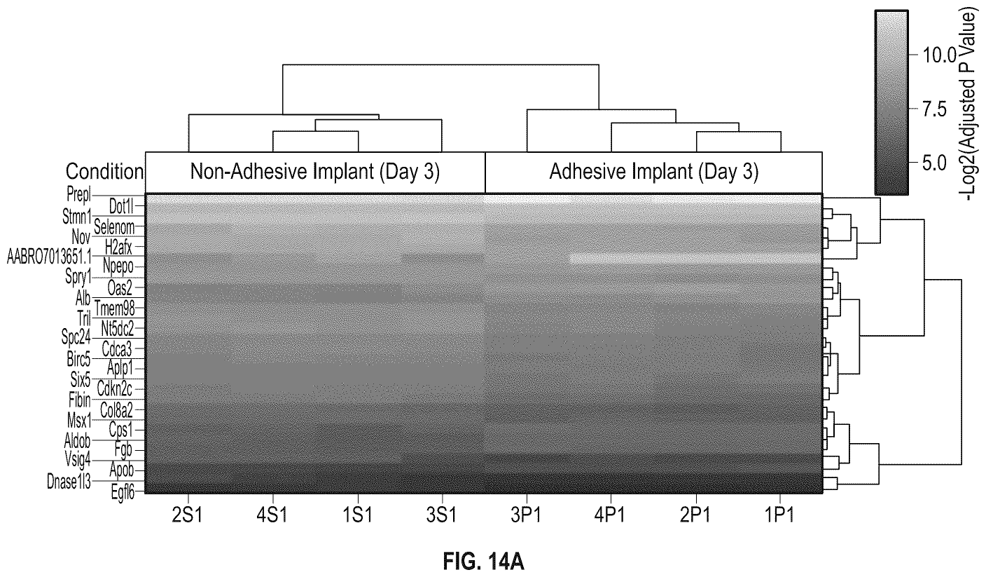

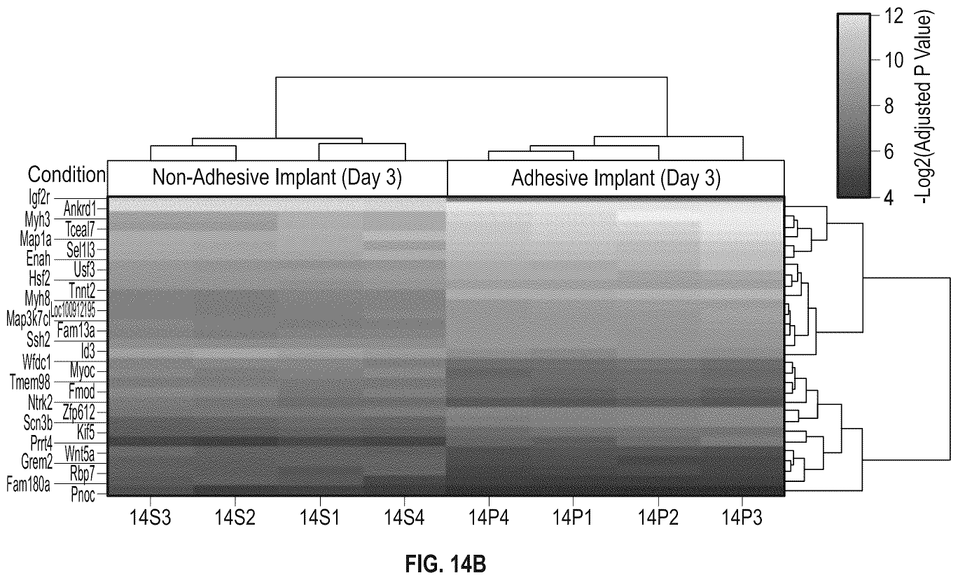

Bulk RNA sequencing was performed of implant-abdominal wall interfaces for both adhesive and non-adhesive implants on day 3 and day 14 post-implantation to further investigate gene-expression differences (FIG. 13). Principal component analysis (PCA) showed separate clusters of samples for the adhesive and non-adhesive implant-tissue interfaces on each time point, which indicated distinct transcriptomic profiles (FIG. 13A,13D). In addition, differential gene expression analysis of the non-adhesive versus adhesive implant-tissue interfaces revealed moderate differences for day 3 post-implantation with 40 down-regulated and 33 up-regulated genes as displayed on the Volcano plot (FIG. 13B) and heatmap (FIG. 14A). On day 14 post-implantation, 513 significantly differentially expressed genes were identified, with 357 down-regulated and 156 up-regulated (FIG. 13E and FIG. 14B) for the non-adhesive implant-tissue interface compared to the adhesive implant-tissue interface.

To explore the functional implications of the differentially expressed genes, Gene Ontology (GO) enrichment analysis was performed. On day 3 post-implantation, it was discovered that regulation of interferon production and striated muscle tissue development were enriched for the non-adhesive implant-tissue interface, which indicated inflammatory and fibrosis processes, whereas cell proliferation and growth processes were enriched for the adhesive implant-tissue interface (FIG. 13C). Similar analysis for day 14 post-implantation samples demonstrated that mostly fibrosis-associated processes were highly enriched for the non-adhesive implant-tissue interface, such as muscle cell differentiation, myofibril assembly, and muscle structure development, whereas vasculature formation, neurogenesis, and proliferation were primarily enriched for the adhesive implant-tissue interface (FIG. 13F).

FIG. 13A depicts a principal component analysis (PCA) plot illustrating the variances of the adhesive (red dots, n=4) and the non-adhesive (black dots, n=4) implant-tissue interface dataset collected on day 3 post-implantation to the abdominal wall. FIG. 13B is a volcano plot displaying the gene expression profiles when comparing the adhesive and the non-adhesive implant-tissue interfaces collected on day 3 post-implantation to the abdominal wall. Colored (blue and red) data points represent genes that meet the threshold of fold change (FC) above 1 or under −1, False Discovery Rate (FDR)<0.05. FIG. 13C depicts top 5 enriched processes from Gene Ontology (GO) enrichment analysis of differentially expressed genes in the non-adhesive (red) and the adhesive (blue) implant-tissue interfaces collected on day 3 post-implantation to the abdominal wall. FIG. 13D is a PCA plot illustrating the variances of the adhesive (red dots, n=4) and the non-adhesive (black dots, n=4) implant-tissue interface dataset collected on day 14 post-implantation to the abdominal wall. FIG. 13E is a volcano plot displaying the gene expression profiles when comparing the adhesive and the non-adhesive implant-tissue interfaces collected on day 14 post-implantation to the abdominal wall. Colored (blue and red) data points represent genes that meet the threshold of FC above 1 or under −1, FDR<0.05. FIG. 13F depicts the top 5 enriched processes from GO enrichment analysis of differentially expressed genes in the non-adhesive (red) and the adhesive (blue) implant-tissue interfaces collected on day 14 post-implantation to the abdominal wall.

FIG. 14A and FIG. 14B are bi-clustering heatmaps that visualize the expression profiles of the top 30 differentially expressed genes sorted by their adjusted P value by plotting their log2 transformed expression values in samples 3 days (FIG. 14A) and 14 days (FIG. 14B) post-implantation. Dendrograms were drawn from Ward hierarchical clustering.

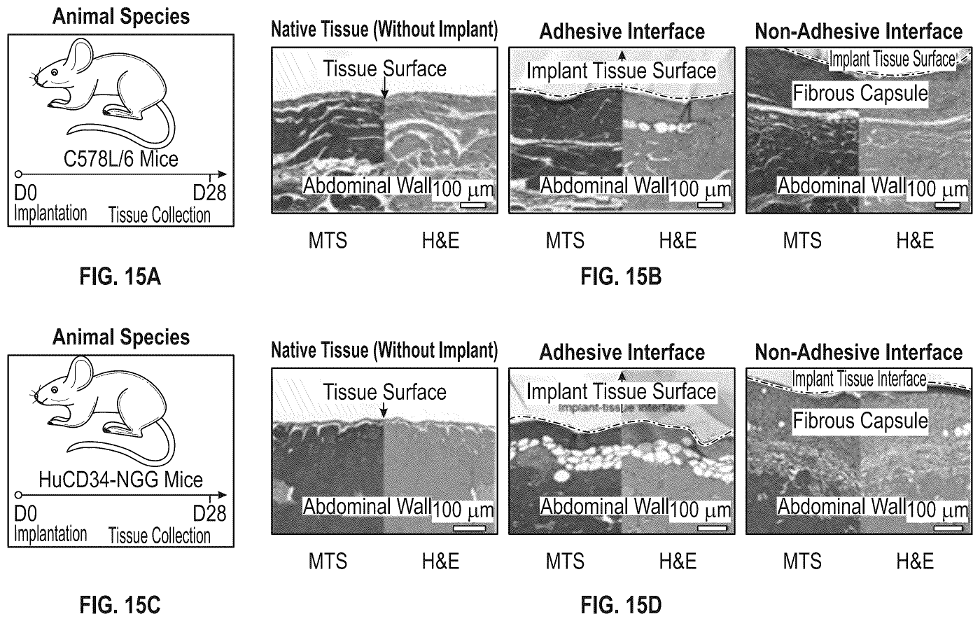

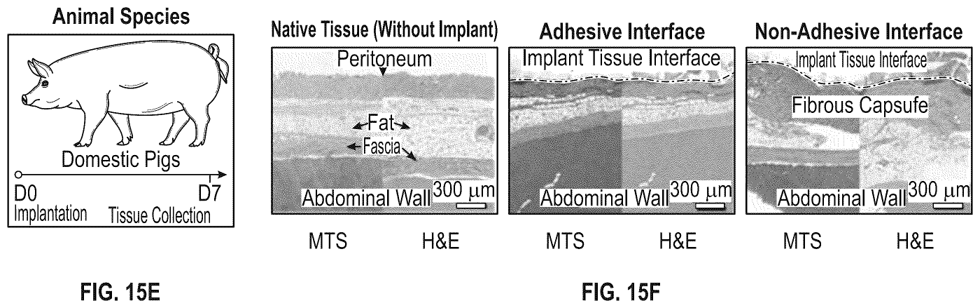

In order to test diverse animal models, embodiments of adhesive and non-adhesive implants were implanted on the abdominal wall surface of immunocompetent C57BL/6 mice and HuCD34-NCG humanized mice (FIG. 15A and FIG. 15C). Immunocompetent C57BL/6 mice are known to produce fibrosis and foreign body reactions similar to those observed in human patients, while HuCD34-NCG humanized mice typically provide human-like immune responses.

Histological analysis showed that the adhesive implant-tissue interface of this example exhibited no observable formation of a fibrous capsule, compared to the native tissue on day 28 post-implantation in both the C57BL/6 (FIG. 15B) and HuCD34-NCG (FIG. 15D) mouse models of this example. In contrast, the non-adhesive implant-tissue interface showed substantial formation of a fibrous capsule in both mouse models of this example (FIG. 15B and FIG. 15D).

To test human-scale anatomy, an embodiment of an adhesive implant and non-adhesive implants were implanted in porcine models (FIG. 15E). Macroscopic observations demonstrated that the adhesive implant of this example maintained stable integration with the surface of the porcine abdominal wall on day 7 post-implantation in vivo. Histological analysis showed that the adhesive implant formed conformal integration with the tissue surface without observable formation of the fibrous capsule on the implant-tissue interface on day 7 post-implantation for the abdominal wall (FIG. 15F). In contrast, the non-adhesive implant-tissue interface exhibited substantial formation of a fibrous capsule (FIG. 15F), which was consistent with rodent models.

In vivo intraperitoneal implantation in porcine model: Female domestic pigs (female, 50 kg, 20 weeks) were placed in dorsal recumbency, and the abdominal region was clipped and prepared aseptically. A blade was used to incise the ventral midline and extended using electrocautery when indicated. The linea alba was incised and the peritoneum bluntly entered, with the incision extended to match the skin incision. The small intestine was exteriorized and moist lap sponges were used for isolation. Then, an embodiment of an adhesive implant or non-adhesive implant was applied and adhered to the surface of the abdominal wall and small intestine (n=4 for each group). The small intestine was thoroughly lavaged and returned to the abdomen. The entire abdominal cavity was lavaged and suctioned, and then the entire abdominal cavity was lavaged and suctioned. Then the celiotomy incision was closed. On day 7 post-implantation, the animals were humanely euthanized, and the abdominal wall and small intestine of interest were excised and fixed in 10% formalin for 24 hours for histological analyses.

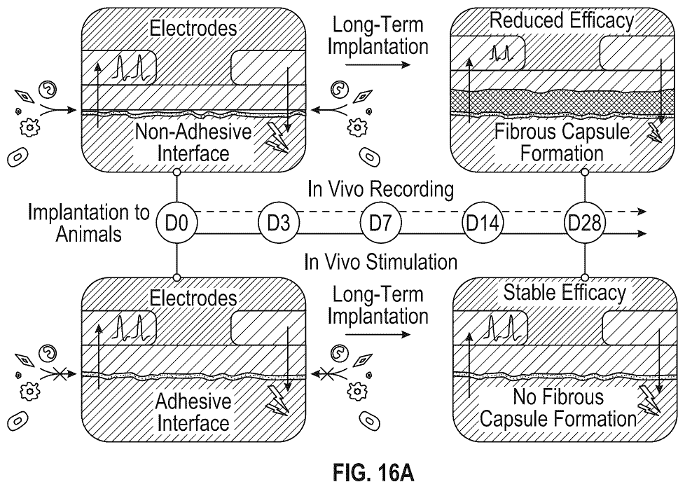

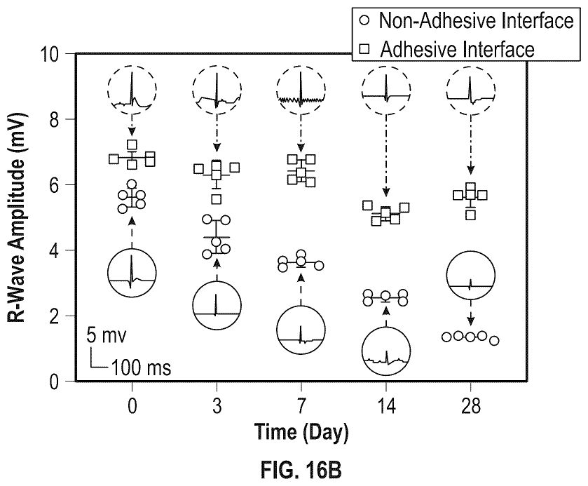

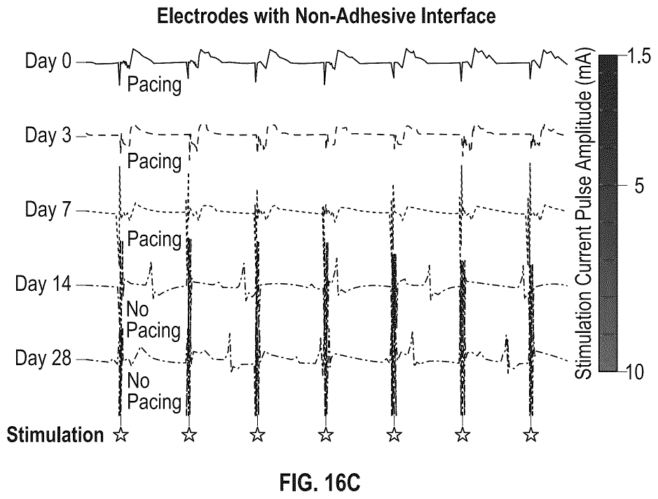

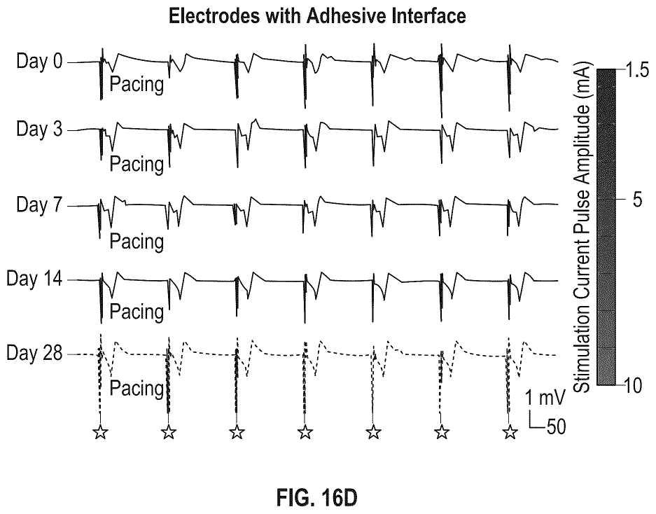

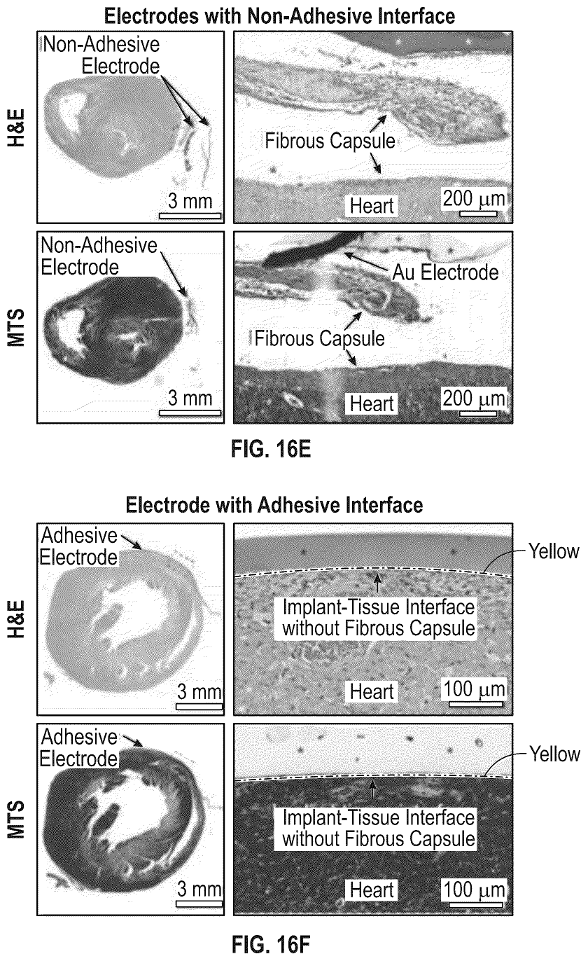

To explore the potential utility of the adhesive implant-tissue interface, long-term in vivo electrophysiological recording and stimulation enabled by the implantable electrodes with the adhesive interface in a rat model was demonstrated (FIG. 16).

For continuous in vivo monitoring and modulation of electrocardiogram, electrodes with the adhesive interface or the non-adhesive interface were implanted to the epicardial surface of animals for electrophysiological recording or stimulation on days 0, 3, 7, 14, and 28 post-implantations (FIG. 16A). The amplitude of R-wave recorded by the electrodes with the adhesive interface was maintained consistently for the duration of the study (28 days post-implantation), whereas the R-wave amplitude recorded by the electrodes with the non-adhesive interface exhibited substantial decrease over time (FIG. 16B). For electrophysical stimulation, the minimal stimulation current pulse amplitude to successfully pace the heart gradually increased until day 7 post-implantation and eventually failed to pace the heart in the longer time points for the electrodes with the non-adhesive interface (FIG. 16C).

In contrast, the electrodes with the adhesive interface exhibited the consistent minimal stimulation current pulse amplitude for pacing and successfully maintained the capability to pace the heart for the duration of the study (28 days post-implantation) (FIG. 16D). These results were consistent with the histological findings from the tissues collected on day 28 post-implantation where the electrodes with the non-adhesive interface showed encapsulation and physical separation from the epicardial surface by thick fibrous capsule (FIG. 16E). In contrast, the electrodes with the adhesive interface showed conformal contact with the epicardial surface without the formation of fibrous capsule on day 28 post-implantation (FIG. 16F).

FIG. 16A includes schematic illustrations for in vivo electrophysiological recording and stimulation via implanted electrodes with the non-adhesive or the adhesive implant-tissue interfaces. FIG. 16B depicts recorded R-wave amplitude via implanted electrodes with the non-adhesive (black) and the adhesive (red) implant-tissue interfaces on days 0, 3, 7, 14, and 28 post-implantations to a rat heart. Inset plots show representative recorded waveforms. FIG. 16C and FIG. 16D are representative epicardial electrocardiograms after stimulation via implanted electrodes with the non-adhesive (FIG. 16C) and the adhesive (FIG. 16D) implant-tissue interfaces on days 0, 3, 7, 14, and 28 post-implantations to a rat heart. FIG. 16E and FIG. 16F are representative histology images stained with hematoxylin and eosin (H&E, top) and Masson's trichrome (MTS, bottom) of the non-adhesive (FIG. 16E) and the adhesive (FIG. 16F) implant collected on day 28 post-implantation to a rat heart. * in images indicates the implant; yellow dotted lines in images indicate the implant-tissue interface. Values in FIG. 16B represent the mean and the standard deviation (n=5; independent samples).

The following procedures were used in this example.

Preparation of adhesive implants: The adhesive layer of the adhesive implant was prepared based on a previously reported method (Yuk, H. et al., Nature 575, 169-174 (2019); Wu, J. et al., Science Translational Medicine 14, eabh2857 (2022)). To prepare an adhesive stock solution, 35 w/w % acrylic acid, 7 w/w % polyvinyl alcohol (M_{w}=146,000-186,000, 99+% hydrolyzed), 0.2 w/w % α-ketoglutaric acid, and 0.05 w/w % N,N′-methylene-bisacrylamide were added into nitrogen-purged deionized water. Then, 30 mg of acrylic acid N-hydroxysuccinimide ester was dissolved per 1 mL of the above stock solution to prepare an adhesive precursor solution.

The precursor solution was poured on a glass mold with a spacer (100-μm thickness) and in a UV chamber (354 nm, 12 W power) for 30 minutes to prepare an adhesive hydrogel. The adhesive hydrogel was thoroughly dried under airflow and a vacuum desiccator to prepare a dry adhesive layer. A mock device of the adhesive implant was introduced by spin-coating a polyurethane resin (HydroThane, AdvanSource Biomaterials) onto the dry adhesive layer.

Preparation of non-adhesive implants: To prepare the non-adhesive implant, the adhesive implant was immersed in a sterile phosphate-buffered saline (PBS) bath overnight. During this process, the adhesive layer of the implant reached the equilibrium swollen state and became non-adhesive by losing the capability to form physical (hydrogen bonds) and covalent (amide bonds) crosslinking with tissues (Chen, X., et al., Proceedings of the National Academy of Sciences 117, 15497-15503 (2020)).

Preparation of implantable electrodes: To prepare the implantable electrodes, the adhesive or non-adhesive layer was integrated onto gold electrodes. The surface of the gold electrode was treated with oxygen plasma for 3 min (30 W power, Harrick Plasma) to activate the surface functionalization, followed by immersing in cysteamine hydrochloride solution (50 mM in deionized water) for 1 hour at room temperature.

After the functionalization, the gold electrode was thoroughly washed with deionized water and dried with nitrogen flow. The functionalized gold electrode was cut into 2-mm diameter circles and placed on the adhesive hydrogel (two electrodes per implant). An electrode lead wire (AS633, Cooner Wire) was connected to the gold electrodes and the polyurethane insulation layer (HydroThane, AdvanSource Biomaterials) was introduced to the gold electrodes. The assembled implant was thoroughly dried under airflow and a vacuum desiccator to prepare the adhesive implantable electrodes. To prepare the non-adhesive implantable electrodes, the adhesive implantable electrodes were immersed in a sterile PBS bath overnight. All samples were prepared in an aseptic manner and were further disinfected under UV for 1 hour before use.

In vitro protein adsorption assay: As a substrate for in vitro protein adsorption assay, a gelatin hydrogel (10 w/v %, 300 g Bloom, Sigma-Aldrich) was used. The adhesive or the non-adhesive implants was cut in 5-mm diameter circles by using a biopsy punch and placed on the gelatin hydrogel. The samples were then incubated in a solution with 5 mg mL⁻¹ fluorescently-tagged albumin (A13101, Thermo Fisher) or fibrinogen (F13191, Thermo Fisher) for 30 minutes. After the incubation, the samples were washed three times with fresh PBS to remove unadhered proteins. The samples were imaged using a confocal microscope (SP8, Leica) with the confocal plane placed at the gelatin hydrogel-implant interface under a pitch model with excitation/emission at 495 nm/515 nm (for albumin) and 495 nm/635 nm (for fibrinogen). The relative fluorescence intensity of absorbed proteins was calculated by using Image J (version 2.1.0).

In vivo intraperitoneal implantation: Female Sprague-Dawley rats (225 to 250 g, 12 weeks, Charles River Laboratories) were used for all in vivo studies. Before implantation, all samples were prepared using aseptic techniques and were further disinfected for 1 hour under UV light. For in vivo intraperitoneal implantation, the animals were anesthetized using isoflurane (2 to 3% isoflurane in oxygen) in an anesthetizing chamber before the surgery, and anesthesia was maintained using a nose cone throughout the surgery. Abdominal hair was removed, and the animals were placed on a heating pad during the surgery. The abdominal wall, colon, or stomach was exposed via a laparotomy. The adhesive implant (10 mm in width and 10 mm in length) was applied to the abdominal wall (n=4 per time point), colon (n=4), or stomach (n=4) surface by gently pressing a surgical spatula or fingertip. The non-adhesive implant (10 mm in width and 10 mm in length) was implanted to the abdominal wall (n=4 per time point), colon (n=4), or stomach (n=4) surface by sutures at the corners of the samples (8-0 Prolene, Ethicon). The abdominal wall muscle and skin incisions were closed with sutures (4-0 Vicryl, Ethicon). On 3 days, 7 days, 14 days, 28 days, and 84 days post-implantation, the animals were euthanized by CO₂ inhalation. Abdominal wall, colon, or stomach tissues of interest were excised and fixed in 10% formalin for 24 hours for histological and immunofluorescence analysis. All animals in the study were survived and kept in normal health conditions based on daily monitoring by the MIT DCM veterinarian staff.

In vivo intrathoracic implantation: For in vivo intrathoracic implantation, the animals were anesthetized using isoflurane (2 to 3% isoflurane in oxygen) in an anesthetizing chamber before the surgery, and anesthesia was maintained using a nose cone throughout the surgery. Chest hair was removed, and endotracheal intubation was performed and connected to a mechanical ventilator (RoVent, Kent Scientific). The animals were placed over a heating pad for the duration of the surgery. The lung or heart was exposed via a thoracotomy and the pericardium was removed using fine forceps for the heart implantation. The adhesive implant (10 mm in width and 10 mm in length) was applied to the lung (n=4) or heart (n=4) surface by gently pressing a surgical spatula or fingertip. The non-adhesive implant (10 mm in width and 10 mm in length) was implanted to the lung (n=4) or heart (n=4) surface by sutures at the corners of the samples (8-0 Prolene, Ethicon). The muscle and skin incisions were closed with sutures (4-0 Vicryl, Ethicon). The animal was ventilated with 100% oxygen until autonomous breathing was regained. At 28 days post-implantation, the animals were euthanized by CO₂ inhalation. Lung or heart tissues of interest were excised and fixed in 10% formalin for 24 h for histological and immunofluorescence analysis. All animals in the study were survived and kept in normal health conditions based on daily monitoring.

In vivo electrophysiological study: Before implantation, the adhesive and the non-adhesive implantable electrodes were prepared using aseptic techniques and were further disinfected for 1 hour under UV. For in vivo epicardial electrode implantation, the animals were anesthetized using isoflurane (2 to 3% isoflurane in oxygen) in an anesthetizing chamber before the surgery, and anesthesia was maintained using a nose cone throughout the surgery. Chest and back hair were removed, and endotracheal intubation was performed and connected to a mechanical ventilator (RoVent, Kent Scientific). The animals were placed over a heating pad for the duration of the surgery. The heart was exposed via a thoracotomy and the pericardium was removed using fine forceps for the epicardial implantation. The adhesive implantable electrodes were applied to the left ventricular surface (n=4) by gently pressing a surgical spatula or fingertip. The non-adhesive implantable electrodes were implanted to the left ventricular surface (n=4) by sutures at the corners of the samples (8-0 Prolene, Ethicon). The lead wire was then tunneled subcutaneously from a ventral exit site close to the left fourth intercostal space to the dorsal side. The dorsal end of the lead wire was inserted through a subcutaneous port. The subcutaneous port was placed by interrupted sutures (4-0 Vicryl, Ethicon) between the shoulder blades of the animal and covered by a protective aluminum cap (VABRC, Instech Laboratories). The muscle and skin incisions were closed with sutures (4-0 Vicryl, Ethicon). The animal was ventilated with 100% oxygen until autonomous breathing was regained.

On days 0, 3, 7, 14, and 28 post-implantations, each animal was anesthetized and connected to the data acquisition hardware (PowerLab, AD Instrument) and software (LabChart Pro 7, AD Instrument) for electrophysiological recording and stimulation by the implanted electrodes. For electrophysiological recording, the data acquisition hardware was connected to the implanted electrodes through the dorsal subcutaneous port. Epicardial signals were recorded to evaluate the R-wave amplitude. For electrophysiological stimulation, an external stimulator (FE180, AD Instrument) was connected to the implanted electrodes through the dorsal subcutaneous port. Unipolar rectangular current pulses (0.5 ms, 0-3 mA, 5-7 Hz) were used for continuous ventricular pacing while the surface electrocardiogram (ECG) was monitored to evaluate the capture threshold. At 28 days post-implantation, the animals were euthanized by CO₂ inhalation. Heart tissues of interest were excised and fixed in 10% formalin for 24 hour for histological analysis. All animals in the study were survived and kept in normal health conditions based on daily monitoring.

Immunofluorescence analysis: The expression of targeted markers (αSMA, Collagen I, CD68, CD3, CD206, iNOS, Vimentin, Collagen III, Neutrophil Elastase) was analyzed after the immunofluorescence staining of the collected tissues. Before the immunofluorescence analysis, the paraffin-imbedded fixed tissues were sliced and prepared into slides. The slides were deparaffinized and rehydrated with deionized water. Antigen retrieval was performed using the steam method during which the slides were steamed in IHC-Tek Epitope Retrieval Solution (IW-1100) for 35 minutes, and then cooled for 20 minutes. Then the slides were washed in three changes of PBS for 5 minutes per each cycle. After washing, the slides were incubated in primary antibodies (1:200 mouse anti-αSMA (ab7817, Abcam); 1:200 mouse anti-CD68 (ab201340, Abcam); 1:100 rabbit anti-CD3 (ab5690, Abcam); 1:200 rabbit anti-collagen-I (ab21286, Abcam)); 1:1000 rabbit anti-CD206 (ab64693, Abcam); 1:500 mouse anti-vimentin (ab8978, Abcam); 1:1000 rabbit anti-collagen III (ab283694, Abcam); 1:2000 rabbit anti-iNOS (ab283655, Abcam); 1:200 mouse anti-iNOS (GTX60599, GeneTex); 1:50 rabbit anti-neutrophil elastase (bs-6982R, Bioss) diluted with IHC-Tek Antibody Diluent for 1 hour at room temperature. The slides were then washed three times in PBS and incubated with Alexa Fluor 488 labeled anti-rabbit or anti-mouse secondary antibody (1:200, Jackson Immunoresearch) or Alexa Fluor 594 labeled donkey anti-mouse secondary antibody (1:200, Jackson Immunoresearch) for 30 minutes. The slides were washed in PBS and then counterstained with propidium iodide solution for 20 min. A laser confocal microscope (SP8, Leica) was used for image acquisition. ImageJ (version 2.1.0) was used to quantify the fluorescence intensity of expressed antibodies. All the images were transformed into 8-bit binary images, and the fluorescence intensity was calculated with normalized analysis. All analyses were blinded with respect to the experimental conditions.

Luminex quantitation analysis: On days 1, 3, and 7 post-implantations, the abdominal muscle wall of interest was collected. The collected samples were snap-frozen in liquid nitrogen and homogenized on a TissueLyser LT (Qiagen) following the manufacturer's instructions. A Luminex multiplex assay was used to measure the concentrations of immune response-related cytokines and chemokines (RECYTMAG-65K, Milliplex).

qPCR analysis: RNA was isolated from the samples snap-frozen in liquid nitrogen immediately after excision using the TRIzol protocol (Invitrogen). All samples were homogenized and normalized by loading 1 μg of total RNA in all cases for reverse transcription using a SuperScript First Strand cDNA Synthesis Kit (Invitrogen). Complementary DNA (1:20 dilution) was amplified by qPCR with the following primers: Mrc1 (5′-AACTTCATCTGCCAGCGACA-3′; reverse: 5′-CGTGCCTCTTTCCAGGTCTT-3′), Tgfb1 (5′-AGTGGCTGAACCAAGGAGAC-3′; reverse: 5′-CCTCGACGTTTGGGACTGAT-3′), Nos2 (5′-TGGTGAGGGGACTGGACTTT-3′; reverse: 5′-CCAACTCTGCTGTTCTCCGT-3′), Cd86 (5′-AGACATGTGTAACCTGCACCAT-3′; reverse: 5′-TACGAGCTCACTCGGGCTTA-3′), Col1a1 (5′-ATGCTGAATCGTCCCACCAG-3′; reverse: 5′-ATGTCCCGGCAGGATTTGAA-3′), Acta2 (5′-GGATCAGCGCCTTCAGTTCT-3′; reverse: 5′-AGGGCTAGAAGGGTAGCACA-3′), Il2 (5′-CCAAGCAGGCCACAGAATTG-3′; reverse: 5′-TCCAGCGTCTTCCAAGTGAA-3′), S100a8 (5′-CGAAGAGTTCCTTGTGTTGGTG-3′; reverse: 5′-AGCTCTGTTACTCCTTGTGGC-3′), Ly6c (5′-ACCTGGTCACAGAGAGGAAGT-3′; reverse: 5′-AGCAGTTAGCATTAAGTGGGACT-3′), Il10 (5′-TTGAACCACCCGGCATCTAC-3′; reverse: 5′-CCAAGGAGTTGCTCCCGTTA-3′), Cd11b (5′-GACTCCGCATTTGCCCTACT-3′; reverse: 5′-GCTGCCCACAATGAGTGGTA-3′), and glyceraldehyde-3-phosphate dehydrogenase (GAPDH) (5′-CACCATCTTCCAGGAGCGAG-3′; reverse: 5′-CCACGACATACTCAGCACCA-3′). Samples were incubated for 10 minutes at 95° C. for 15 seconds and at 60° C. for 1 minute in the real-time cycler Agilent MX3000P. GAPDH was used as the reference gene for normalization and analysis. The comparative CT (ΔΔCT) method was used for relative quantification of gene expression.

RNA-seq analysis: RNA extraction, library preparations, and sequencing reactions were conducted at GENEWIZ, LLC. Total RNA was extracted using the Qiagen RNeasy Plus Universal mini kit following the manufacturer's instructions (Qiagen). Extracted RNA samples were quantified using Qubit 2.0 Fluorometer (Life Technologies) and RNA integrity was checked on Agilent TapeStation 4200 (Agilent Technologies). RNA sequencing libraries were prepared using the NEBNext Ultra RNA Library Prep Kit for Illumina following the manufacturer's instructions (NEB). Briefly, mRNAs were first enriched with Oligo(dT) beads. Enriched mRNAs were fragmented for 15 min at 94° C. First-strand and second-strand cDNAs were subsequently synthesized. cDNA fragments were end-repaired and adenylated at 3′ends, and universal adapters were ligated to cDNA fragments, followed by index addition and library enrichment by limited-cycle PCR. The sequencing libraries were validated on the Agilent TapeStation (Agilent Technologies), and quantified by using Qubit 2.0 Fluorometer (Invitrogen) as well as by quantitative PCR (KAPA Biosystems). The sequencing libraries were clustered on 1 lane of a flow cell. After clustering, the flowcell was loaded on the Illumina HiSeq 4000 instrument and the samples were sequenced using a 2×150 bp Paired End (PE) configuration. Image analysis and base calling were conducted by the HiSeq Control Software (HCS). Raw sequence data (.bel files) generated from Illumina HiSeq were converted into fastq files and de-multiplexed using Illumina's bcl2fastq 2.17 software. One mismatch was allowed for index sequence identification.

Read quality was evaluated using FastQC and data were pre-processed with Cutadapt (Martin, M. et al., EMBnet. Journal 17, 10-12 (2011) for adapter removal following best practices. Gene expression against the mRatBN7.2 transcriptome (Ensembl release 104) (Cunningham, F. et al., Nucleic Acids Research 47, D745-D751 (2019)) was quantified with STAR (Dobin, A. et al., Bioinformatics 29, 15-21 (2013)) and featureCounts (Liao, Y. et al., Bioinformatics 30, 923-930 (2014)). Differential gene expression analysis was performed using DESeq2 (Love, M. I. et al., Genome Biology 15, 550 (2014)), while ClusterProfiler (Yu, G. et al., Omics: A Journal of Integrative Biology 16, 284-287 (2012)) was utilized for functional enrichment investigations. Genes with log2|Fold Change|≥1 and False Discovery Rate (FDR)≤0.05 were considered statistically significant.

We claim:

1. A method of implanting a biomaterial or a device in a patient, the method comprising:
arranging an adhesive composition between and in contact with (i) the biomaterial or the device, and (ii) a surface of a tissue of the patient;
wherein the adhesive composition is configured to form at the surface of the tissue a conformal interface that reduces or minimizes infiltration of inflammatory cells at the conformal interface.

2. The method of claim 1, wherein the reduction or the minimization of the infiltration of inflammatory cells prevents the formation of a fibrous capsule at the conformal interface for at least 28 days after the implanting of the biomaterial or the device.

3. The method of claim 1, wherein the device comprises an electrode.

4. The method of claim 3, wherein bi-directional electrical communication between the electrode and the tissue is maintained for at least 28 days after the implanting of the electrode.

5. The method of claim 4, wherein the tissue comprises heart tissue of the patient's heart, and an R-wave of the patient's heart recorded by the electrode at least 28 days after the implanting of the electrode has an amplitude that is 0% to about 5% less than an initial amplitude of an initial R-wave of the patient's heart recorded by the electrode within 24 hours after the implanting of the electrode.

6. The method of claim 4, wherein the tissue comprises heart tissue of the patient's heart, and a minimal stimulation current pulse amplitude for pacing emitted by the electrode paces the patient's heart for at least 28 days after the implanting of the electrode.

7. The method of claim 1, wherein the tissue comprises abdominal wall tissue, colon tissue, stomach tissue, lung tissue, or heart tissue.

8. The method of claim 1, wherein the arranging of the adhesive composition comprises:
providing the adhesive composition, wherein the adhesive composition is affixed to the biomaterial or the device; and
contacting the surface of the tissue with the adhesive composition.

9. The method of claim 1, wherein the arranging of the adhesive composition comprises:
disposing the adhesive composition on the surface of the tissue; and
contacting the adhesive composition with the biomaterial or the device.

10. The method of claim 1, wherein the adhesive composition comprises a crosslinked polymeric composition.

11. The method of claim 1, wherein the adhesive composition comprises:
(i) one or more hydrophilic polymers, one or more zwitterionic polymers, or a combination thereof;
(ii) one or more tissue coupling groups;
(iii) one or more crosslinkers; or
(iv) a combination thereof.

12. The method of claim 11, wherein the one or more hydrophilic polymers are selected from the group consisting of polyethylene glycol, polyacrylic acid, polyacrylamide, polyvinyl alcohol, polyhydroxy ethyl methacrylate, polyethylene glycol, polyurethane, casein, albumin, gelatin, chitosan, dextran, hyaluronic acid, alginate, cellulose, polyvinyl pyrrolidone, polystyrene sulfonate, collagen, alginic acid, pectin, and a combination thereof.

13. The method of claim 12, wherein the alginate and the cellulose are oxidized alginate and oxidized cellulose, respectively.

14. The method of claim 11, wherein the one or more zwitterionic polymers are selected from the group consisting of poly(phospobetaine), poly(carboxybetaine), poly(sulfobetaine), and a combination thereof.

15. The method of claim 11, wherein the one or more tissue coupling groups is selected from the group consisting of an amine coupling group, a thiol coupling group, a cysteine coupling group, an N-acetyl-cysteine coupling group, a boronate ester coupling group, and a combination thereof.

16. The method of claim 15, wherein the amine coupling group is selected from the group consisting of an N-hydroxysuccinimide ester (PAAc-co-NHS ester), a PAAm-co-NHS, an N-Hydroxysuccinimide (NHS) PEG, a poly(L-lactide-co-glycolide)-NHS, a poly(D,L-lactide)-polyethylene glycol-CO-NHS, a poly(N-isopropylacrylamide) N-hydroxysuccinimide terminated, an aldehyde, an imidoester, an epoxide, an isocyanate, a catechol, and a combination thereof.

17. The method of claim 15, wherein the thiol coupling group is selected from the group consisting of alginate, albumin, fibrinogen, collagen, chitosan, gelatin, and a combination thereof; and wherein the cysteine coupling group is selected from the group consisting of fibrinogen, collagen, and a combination thereof.

18. The method of claim 15, wherein the one or more boronate ester coupling groups is selected from the group consisting of acrylamide, N-isopropylacrylamide, polyvinyl alcohol (PVA), alginate, cellulose, and a combination thereof.

19. The method of claim 11, wherein the one or more crosslinkers are selected from the group consisting of gelatin methacrylate, hyaluronic acid methacrylate, oxidized methacrylic alginate, polycaprolactone diacrylate, N,N′-bis(acryloyl) cystamine, N,N′-methylenebis(acrylamide), polyethylene glycol diacrylate, polyethylene glycol dimethacrylate, and a combination thereof.

20. An adhesive composition configured to form at a surface of a tissue a conformal interface that reduces or minimizes infiltration of inflammatory cells at the conformal interface.
